SEQUENCE LISTING (40 sequences)

SEQ ID NO: 1 - protein, 95 aa, Cali mammarenavirus
MGLRYSKEVRKRHGDEDVVGRVPMTLNLPQGLYGRFNCKSCWFVNKGLIRCKDHYLCLGCLTKMHSRGNLCEICGHSLPTKMEFLESPSAPPYEP

SEQ ID NO: 2 - protein, 2194 aa, Cali mammarenavirus
MEEYVFELKDIVRKWVPEWEELSEQKNNVLAQVKDRAITIEGLKLLSMLVEVDSCKKHSCKHNTKMTVNAILRELRVTCPTLPDVTPDGYCMVGDVLILLEVFVRTSQEAFEKKYNQDFLKLLQLSSDLKRQNITLVPVIDGRSSYYVEFVPDWVVERLRWLLLKLMDGLRTSGEEVEELEYERLISSLSSLENQSLGLESLLAVKERGLPYKVRLEKALMSGINNKLTTDQCRTKIMEIFQQFKMLQLAGQLDRKLQATDREDMISRLQNHEFIQCSVKDVPKSEIRLCEFCSVHILGIIGQLRQSEVKHSSTESREYFRVLSICNKIKSQKVFNTRRNTMLVLDLIMYNILCDLDKSSPGAVFREVLLMQGLPSVNDRLINVDFLMEQITKKFIKNPNWLEKAKKRLSSVCGELPLDDILPLLREPDVEYYFNLKTSVLDEWGAKPCLQYKTKSQCMCGGRPGRGQPDYTIMGESEFEELLKTLSSLSLSLINSMKTAAVPKMKVNNADEFYGKVYCDEVFFQRFGEGGSLTLLYQKTGERSRCYAVAYRSKSGGLYETKASFYCDPKRFFLPIFSADVIQRTCVEMLSWLDFMSQPLLDSVSDLLRRLILCILCTPSKRIQVYLQGFRYYIMAFVNEVHFKELFEKLKVVMLTPSEWQTAMLIDDLILLVLSNSREEDMAKIFKFVLNVSYLCHFITKETPDRLTDQIKCFEKFLEPKLKFDSVLVNPSNSMELPTEEEEKMVHDIERLLGKKLESKCEGRPGLNKDVLSVCLSLFNSSSLEVKPLLPCDPMTPSFTSTALDMSSNKSVVVPKLNEVGEVITEYDYSSIVSAVVVEMIEHFKTKGKYKLDPKEVNFKILKRLSSLIQIKKESIEPDGVEELLSEDQGDCLKEIETRVAKVLSKVDTNVKTNLKTSCPLERLWPKSTMVVIKRETSLHDVKDFDYSLFSAEVYEDLVNLIYEDVTARSVYFADRLMNPCPLEFLIKNLTLKAYKEADYFECFKYILIASDYDNRVGRYDHKSRSRLGFTDAALQIRETSRISSRESNSESIAKRLDQSFFTNSSLRNLCFYSDESPTERSGVSTNVGRLKFGLSYKEQVGGNRELYVGDLNTKLTTRLIEDYSESLMQNMRYTCLNNEKEFERALLDMKSVVRQSGLAVSMDHSKWGPHMSPVIFAALLKGLEFKLKDGSEVPNAAVINILLWHIHKMVEVPFNVVEAYMKGFLKRGLGMMDKGGCTIAEEFMFGYFEKGKVPSHISSVLDMGQGILHNTSDLYGLITEQFINYALELCYGARFISYTSSDDEIMLSLNEGFKFKDRDELNVELVLDCMEFHYFLSDKLNKFVSPKTVVGTFASEFKSRFFIWSQEVPLLTKFVAAALHNIKAKAPNQQADTIDTILDQCVANGVSIEVVGAIAKRTNSMIIYSGFPNDPFLCLEEMDVLDWVNGSRGYRLQRSIETLFPDDLLLSIIRKACRKIFYKIQSGALEESYIVTTLQQSPDDCLKQLLETCDVETEAIEDALNIRWLNLRVHGDLRLVLRTKLMSTTRTVQREEIPSLVKSVQSKLSKNYVRGAKKILADAINKSAFQSSIASGFIGVCKSMGSKCVRDGKGGFKYIRDITSKIILHRDCHFCNQRKGVYCKAALGEVSEYSRPLIWDYFALVLTNACELGNWVFQKAEVPKIVTHLNNPNHFWPIKPSTHSELEDKVGINHILYSIRRNFPTLFDEHISPFLSDLNMLRLSWVQRIKFLDLCVAIDITSECLGIVSHIIKHRREELYIVKQNELAMSHSRESHPLERGFNLEPEEVCTNFLIQILFESMLVPVIMSTSQFKKYFWFGELELLPNNAQHDLKQLTQFICDCKKNNTSRTMNLDDLDVGFVSSKLILSCVNLNISVFINELDWVNRDNYENIEQLILASPSEVIPIELNLTFSHKRVSHKFRYERSTNYILKLRFLIERESLLDSLDSDGYLLLNPHSVEYYVSQSSGNHISLDGVSLLVLNPLINGKDVLDFNDLLEGQDIHFKSRSTVFQKVRIDLKNRFKDLKNKFSYKLIGPDVGMQPLILEGGLIKEGNRVVSRLEVNLDSKVVIIALEALEPEKRPRFIANLFQYLSSAQSHNKGISMNEQDLRLMIENFPEVFEHMLHDAKDWLNCGHFSIIRSKTLGSVMIADETGPFKIKGIRCRKLFEDNESVEIE

SEQ ID NO: 3 - protein, 561 aa, Cali mammarenavirus
MSDNIPSFRWVQSLRRGLSNWTHPVKADVLSDTRALLSALDFHKVAQVQRMMRKDKRTDSDLTKLRDMNKEVDALMNMRSIQRDNVLKVGGLAKEELMELASDLDKLRKKVTRTESLSQPGVYGGNLTNTQLEQRAEILRSMGFANARPTGNRDGVVKIWDIKDNTLLINQFGSMPALTIACMTEQGGEQLNDVVQALSALGLLYTVKFPNMTDLEKLTQQHSALKIISNEPSAINISGYNLSLSAAVKAAACMIDGGNMLETIQVKPSMFSTLIKSLLQIKNREGMFVSTTPGQRNPYENLLYKICLSGDGWPYIGSRSQVQGRAWDNTTVDLDSKPSAIQPPVRNGGSPDLKQIPKEKEDTVVSSIQMLDSKATTWIDIEGTPNDPVEMAIYQPDTGNYIHCYRFPHDEKSFKEQSKYSHGLLLKDLADAQPGLISSIIRHLPQNMVFTAQGSDDIISLFEMHGRRDLKVLDVKLSAEQARTFEDEIWERYNLLCTKHKGLVIKKKKKGAAQTTANPHCALLDTIMFDATVTGWVRDQKPMRCLPIDTLYRNNTDLINL

SEQ ID NO: 4 - protein, 508 aa, Cali mammarenavirus
MGQVVTLIQSIPEVLQEVFNVALIIVSTLCIIKGFVNLMRCGLFQLITFLILAGRSCDGMMIDRRHNLTHVEFNLTRMFDNLPQSCSKNNTHHYYKGPSNTTWGIELTLTNTSIANETTGNFSNIRSLAYGNISNCDKTEEAGHTLKWLLNELHFNVLHVTRHVGARCKTVEGAGVLIQYNLTVGDRGGEVGRHLIASLAQIIGDPKIAWVGKCFNNCSGGSCRLTNCEGGTHYNFLIIQNTTWENHCTYTPMATIRMALQKTAYSSVSRKLLGFFTWDLSDSTGQHVPGGYCLEQWAIVWAGIKCFDNTVMAKCNKDHNEEFCDTMRLFDFNQNAIKTLQLNVENSLNLFKKTINGLISDSLVIRNSLKQLAKIPYCNYTKFWYINDTITGRHSLPQCWLVHNGSYLNETHFKNDWLWESQNLYNEMLIKEYEERQGKTPLALTDICFWSLVFYTITVFLHLVGIPTHRHIIGDGCPKPHRITRNSLCSCGYYKIPKKPYKWVRLGK

SEQ ID NO: 5 - protein, 326 aa, Artificial Sequence
  sigma C protein
MAALTPSQRREVVGLILSLTSNANTSCGDLAPILERLVRLDSTVKLLTESVGVLSQKVSAFESSLKDTNSSLSKVTASMTELSGELHQLHASLGDVDTSVLDMSATLTKHGNLITALQTSVQNNANAISNLKSSVSALGLSVTDIDGRLKAVESGSGSPLKFSTPLKLDGGVVSLDMDPYFCSDNHVLTSYSTDAQLMQFQWLARGADGSSGSVDMLVNAHCHGRRTDYMMSTTENLTITGNSTSLVFNLDYITKPPTDMSRLIPRAGFQAASFPVDVSFTRDDTTHAYQVYGTFTSPRVFKITFLTGGAGTANLRFLTVRTGIDT

SEQ ID NO: 6 - DNA, 978 bp, Avian orthoreovirus
atggccgctttaacacctagccagaggagggaggtggtgggcctcattttatctttaacctccaatgccaacacctcttgtggagatttagcccccattttagagagactggtgaggctggacagcaccgtgaaactgctgaccgaatccgtgggcgtgctgagccagaaggtgagcgctttcgagagctctttaaaggacaccaactcctctttaagcaaagtcacagccagcatgacagaactgtccggcgagctgcaccaactgcacgcctctttaggcgacgtggataccagcgtgctggacatgtccgccactttaaccaagcacggaaatttaatcaccgctttacagaccagcgtgcagaacaacgctaacgccatctccaatttaaagagcagcgtgtccgctttaggactgagcgtgacagacatcgacggtcgtctgaaggctgtggagagcggcagcggcagccctttaaagttctccacccctctgaagctggacggcggcgtggtgtctttagacatggacccctacttctgcagcgacaaccacgtgctgaccagctattccaccgacgcccagctgatgcagttccagtggctcgctagaggcgctgatggcagctccggaagcgtcgacatgctggtgaacgcccactgtcatggcagaaggaccgactacatgatgtccaccaccgagaatttaaccatcaccggcaacagcaccagcctcgtgttcaatttagactacatcaccaagccccccaccgacatgtccagactgattcctagagctggcttccaagccgccagcttccccgtggatgtgagcttcacaagagacgacaccacacacgcctaccaagtgtacggcaccttcaccagccctcgtgtgttcaagatcacctttttaactggtggcgctggcaccgccaatttaagatttttaacagtgaggaccggaatcgacacc

SEQ ID NO: 7 - protein, 367 aa, Avian orthoreovirus
MEVRVPNFHSFVEGITSSYIRAPACWNAKTMWDVETFHLPDVIKVGNAYCCSQCCGVLYYGAPPSDGNYFPHHKCHQQQYRSDTPLLRYVRIGRTTEHLLDQYAVTLQTIADYYDETSHRVADDSEEDTIAALDIVTRTESIRSDQAVDADFWTYPLERRSDDSRRDIAASIWTMIDASARSFTLPDCLVSPSLHSRHVFDQMLTTTSIYDVAASGKTARFSPMVAALPQRAAGPITLPDADPLDGVATFWSPQFALSPMIGGVGITGQYARESYHHVGHPVIGSGKKVSHYRNLFMDAWRGWSKSSFTCAAGLEPAECESRLRGHARTMLGRSLPGVCDCGSEAQPRTAPSSLQKATKLTFMECGW

SEQ ID NO: 8 - DNA, 1104 bp, Avian orthoreovirus
atggaggtgagggtgcccaacttccactccttcgtcgagggcatcacctcctcctatatcagagcccccgcttgttggaacgccaagaccatgtgggatgtggagacctttcatttacccgacgtgatcaaggtgggcaacgcctactgttgctcccagtgctgcggcgtgctgtactacggagctccccccagcgatggcaactacttcccccaccacaagtgccaccagcagcagtatcgttccgacacccctttactgagatacgtgagaatcggaaggaccaccgagcatttactggatcagtacgccgtgactttacagaccattgccgactattacgacgagacctcccatcgtgtggctgatgacagcgaggaggacaccatcgccgctttagatatcgtgactcgtaccgagagcatcagaagcgaccaagctgtggacgccgacttctggacctaccctttagagaggaggagcgatgactctcgtagggatatcgccgccagcatctggaccatgatcgacgccagcgctcgtagcttcactttacccgactgtctggtgagccctagcctccactctcgtcacgtgttcgaccagatgctgaccacaaccagcatctacgatgtggccgcttccggcaagacagctcgtttctcccccatggtggccgctctgcctcaaagagccgctggccccatcactttacccgatgccgaccctctggacggcgtggctaccttctggagcccccagttcgccctctcccctatgatcggcggcgtgggcattaccggacagtacgctcgtgagtcctaccaccacgtgggccaccccgtgatcggaagcggcaagaaggtctcccactatcgtaatttattcatggatgcttggagaggatggagcaagtcctccttcacatgtgccgccggtttagaacccgctgaatgcgagagcagactgaggggccatgctcgtaccatgctgggcagatctttacccggcgtgtgtgactgcggcagcgaagctcagcctaggaccgcccctagctctttacagaaggccaccaagctgaccttcatggagtgtggctggtac

SEQ ID NO: 9 - skipped

SEQ ID NO: 10 - protein, 367 aa, Avian orthoreovirus
MEVRVPNFHSFVEGITSSYIRAPACWNAKTMWNIETFHLPDVIKVGNAYCCSQCCGVLYYGAPPSDGNYFPHHKCHQQQYRSDTPLLRYVRIGRTTEHLLDQYAVTLQTIADYYDEASHRVADEPEEDTIAALDIVTRTESIRSDQAVDADFWTYPLERRSDDSRRDIAASIWTMIDASARSFTLPDCLVSPSLHSRHIFDQMLTTTSIYDVAASGKTARFSPMVAALPQRAAGPITLSDADPLDGVATFWSPQFALSPMIGGVGITGQYARESYHHVGHPVIGSGKKVSHYRNIFMDAWRGWSKSSFTCAAGLEPAECESRLRGHARTMLGRSLPGVCDYGPEAQSRTAPSSLQKATKLTVVECGW

SEQ ID NO: 11 - protein, 367 aa, Avian orthoreovirus
MEVRVPNFHSFVEGITSSYIRAPACWNAKTMWDVETFHLPDVIKVGNAYCCSQCCGVLYYGAPPSDGNYFPHHKCHQQQYRSDTPLLRYVRIGRTTEHLLDQYAVTLQTIADYYDEASHRVADESEEDTIAALDIVTRTESIRSDQAVDADFWTYPLERRSDDSRRDIAASIWTMIDASARSFTLPDCLVSPSLHSRHVFNQMLTTTSIYDVAASGKTARFSPMVAAFPQRADGPITLPDADPLDGVATFWSPQFALSPMIGGVGITGQYARESYHHVGHPVIGSGKKVSHYRNLFMDAWRGWSKSSFTCAAGLEPAECESRLRGHARTMLGRSLPEVCDCGSEAQPRTAPSSLQKATKLTFMECGW

SEQ ID NO: 12 - protein, 367 aa, Avian orthoreovirus
MEVRVPNFHSFVEGITSSYIRAPACWNAKTMWDVETFHLPDVIKVGNAYCCSQCCGVLYYGAPPSDGNYFPHHKCHQQQYRSDTPLLRYVRIGRTTEHLLDQYAVALQTIADYYDEASHRVADEPEEDTIAALDIVTRTESIRSDQAVDADFWTYPLERRSDDSRREIAASIWKMIDASARSFTLPDCLVSPSLHSRHVFDQMLTTTSIYDVAASGKTARFSPMVAALPQRTAGPITLADADPFDGVATFWSPQFALSPMIGGVGITGQYARESYHHVGHPVIGGGKKVSHYRNLFMEAWRGWSKSSFTCAAGLEPAECESRLRGHARTMLGRSLPGVCDCGPEAQSRATPSSLQKATKLTVVECGW

SEQ ID NO: 13 - protein, 367 aa, Avian orthoreovirus
MEVRVPNFHSFVEGITSSYIRAPACWNAKTMWDVETFHLPDVIKVGNAYCCSQCCGVLYYGAPPSDGNYFPHHKCHQQQYRSDTPLLRYVRIGSTTEHLLDQYAVALQTIADYYDEASHRVADEAEEDTIAALDIVTRTESIRGDQAVDADFWTYPLERRSDDSRRDIAASIWTMIDASARSFTLPECLVSPSLHSRHIFDQMLTTTSIYDVAASGKTARFSPMVAALPQRTAGPITLPDADPFDGVATFWSPQFALSPMIGGVGITGQYARESYHHVGHPVIGSGKKVSHYRNLFMDAWRGWSKSSFTCAAGLEPAECESRLRGHARTMLGRSLPGVCDCGPEAQSRTAPSSLQKATKLTVVECGW

SEQ ID NO: 14 - protein, 367 aa, Avian orthoreovirus
MEVRVPNFHSFVEGITSSYIRTPACWNAKTTWDVETFHLPDVIKVGNAYCCSQCCGVLYYGAPPPDGNCFPHHKCHQQQYRNDTPLLRYVRIGRTTEHLLDQYAVALQTIADYYEEASLRVANELEENAIAALDIVTRTESIRSDRAVDADFWMYPLERRSDDSRREIAASIWKMIDASARSFTLPDCLVSPSLHSRQVFDQMLTTTSIYDVAASGRTARFSPMVAALPQRTAGPVTLADADPLDGVATFWSPQFALSPMIGGVGITGQYARESYHHVGHPVVGSGKKVSHYRNLFMEAWRGWSKSSFTCAAGLEPAECESRLRGHARTMLGRSLPGVCDCGLEAQSRAAMSSLQKATKLTFMECGW

SEQ ID NO: 15 - protein, 367 aa, Avian orthoreovirus
MEVRVPNFHSFVEGITSSYIRTPACWNAKTTWDVETFHLPDVIKVGNAYCCSQCCGVLYYGVPPPDGNCFPHHKCHQQQYRNDTPLLRYVRIGRTTEHLLDQYAVALQTIADYYEEASLRVANELEENAIAALDIVTRTESIRSDRAVDADFWMYPLERRSDDSRREIAASIWKMIDASARSFTLPDCLVSPSLHSRQVFDQMLTTTSIYDVAASGRTARFSPMVAALPQRTAGPVTLADADPLDGVATFWSPQFALSPMIGGVGITGQYARESYHHVGHPVVGSGKKVSHYRNLFMEAWRGWSKSSFTCAAGLEPAECESRLRGHARTMLGRSLPEVCDCGLEAQSRAAMSSLQKATKLTFMECGW

SEQ ID NO: 16 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRVVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAVKFSPMVATLSQRDAGPVKLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVECGW

SEQ ID NO: 17 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAVKFSPMVATLSQRDAGPVMLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVECGW

SEQ ID NO: 18 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAVKFSPMVATLSQRDAGPVKLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVECGW

SEQ ID NO: 19 - protein, 367 aa, Avian orthoreovirus
MEVRVPNFHSFVEGITSSYLKTPACWNARTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAVKFSPMVATLSQRDAGPVKLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVECGW

SEQ ID NO: 20 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAAKFSPMVATLSQRDAGPVTLANADPAEGVYSFWTSHFAFSPLIGGVGITGQYARESYHHVGHPVIGSGKKASHYRNLFMESWRGWSKSSFACATGMEPAECESRLRGHARTMLGRSLPNVCDDEVAQQSGAVLTSLQKTTKFTVVKCGW

SEQ ID NO: 21 - protein, 366 aa, Avian orthoreovirus
MEVRVPNFHSVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAAKFSPMVATLSQRDAGPVTLANADPAEGAYSFWTSHFAFSPLIGGVGITGQYARESYHHVGHPVIGSGKKASHYRNLFMESWRGWSKSSFACATGMEPAECESRLRGHARTMLGRSLPNVCDDEVAQQSGAVLTSLQKTTKFTVVKCGW

SEQ ID NO: 22 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAAKFSPMVATLSQRDAGPVMLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVKCGW

SEQ ID NO: 23 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWIMIDASSRSLTLPNCLVSPSLHSRSVFGQMQTTTTIYDVAASGKAAKFSPMVATLSQRDAGPVTLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVKCGW

SEQ ID NO: 24 - protein, 367 aa, Avian orthoreovirus
MEVRVPNFHSFVEGITSSYLKTPACWNAQTAWDTVTFHVPDVIIVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASLRSLTLPNCLVSPSLHSRSVFGQMQTTTTIYDVAASGKAAKFSPMVATLSQRDAGPVTLANADPAEGVYSFWTSHFAFSPLIGGVGITGQYARESYHHVGHPVIGSGKKASHYRNLFMESWRGWSKSSFACATGMEPAECESRLRGHARTMLGRSLPNVCDDEVAQQSGAVLTSLQKTTKFTVVKCGW

SEQ ID NO: 25 - protein, 367 aa, Avian orthoreovirus
MEVRVPNFHSFVEGITSSYLQTPACWNAQTAWDTVTFHVPDVIRVGNAYCCSQCCGVLYYGTLPSDGNYFPHHKCHQQQFRTDTPLLRYVRIGRTTEHLLDQYAVALESIAEHYDEISQRMVDEPENDEVTPLDIVTRTESIRSDKAVDPDFWTYPLERRSDDSRRDIASACWKMIDASSRSLTLPNCLVSPSVHSRSVFGQMQTTTTIYDVAASGKAVKFSPMVATLAQRDAGPVTLANADPADGVYSFWTSHFAFSPLIGGVGITGQYARESYHHVGHPVIGSGKKASHYRNLFMEAWRGWSKSAFACATGMEPAECESRLRGHARTMLGRSLPNVCDDDVAQQSGAVLASLQKTTKFTVVECGW

SEQ ID NO: 26 - protein, 367 aa, Avian orthoreovirus
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAAKFSPMVATLSQRDAGPVTLANADPAEGVYSFWTSHFAFSPLIGGVGITGQYARESYHHVGHPVIGSGKKASHYRNLFMESWRGWSKSAFACATGMEPAECESRLRGHARTMLGRSLPNVCDDEVAQQSGAVLTSLQKTTKFTVVKCGW

SEQ ID NO: 27 - protein, 367 aa, Avian orthoreovirus
MEVRVPNFHSFVEGITSSYIRAPACWDSRSAWDSDIFYDPDVIKVGGAYCCTQCCGVLYYGAPPTDGVCFPHHKCHQQLSRVDNPLLRFVKIGRTTEHLLDQYAVILSCIADHYERAAQSQTSMCGGDALMPLDLIVRTESPLTDRPVDSDFWTAPLKRRESDARSDIATAGWRMIDASSRSQTVPDCVVSNLLHTRHVFSQMLTTTTVYDVAVTGKPAKFSPLVAVMPTRGGGTISLTRDNWDHDVEAAWLNGFAFSPIIGGVGISGQFERGACHNYGHPMVGSGKKASHYRNLFMEVCRGWPRSSFTCAVGLEPAECELRLRGQARTMLGRALPDVCDFDETVHTGQSSAPLRRSSKVSFIECGW

SEQ ID NO: 28 - protein, 367 aa, Artificial
  consensus sequence
MEVRVPNFHSFVEGITSSYLKTPACWNAQTAWDTVTFHVPDVIRVGNAYCCSQCCGVLYYGTLPADGNYFPHHKCHQQQYRTDTPLLRYVRIGRTTEHLLDQYAVALESIADHYDEISQRMVDEPENDEVAPLDIVTRTESIRSDKTVDPDFWTYPLERRSDDSRRDIAASCWRMIDASSRSLTLPNCLVSPSLHSRSVFGQMQTTTTIYDVAASGKAAKFSPMVATLSQRDAGPVTLANADPAEGVYSFWTSHFAFSPLIGGVGITGQYARESYHHVGHPVIGSGKKASHYRNLFMESWRGWSKSSFACATGMEPAECESRLRGHARTMLGRSLPNVCDDEVAQQSGAVLTSLQKTTKFTVVECGW

SEQ ID NO: 29 - RNA, 84 nt, Artificial
  5′ UTR sequence
cgcaccggggauccuaggcaucuuugggucacgcuucaaauuuguccaauuugaacccagcucaaguccuggucaaaacuuggg

SEQ ID NO: 30 - RNA, 30 nt, Artificial
  3′ UTR sequence
cgcaccgaggauccuaggcauuucuugauc

SEQ ID NO: 31 - RNA, 51 nt, Artificial
  5′ UTR sequence
cgcaccggggauccuaggcauaccuuggacgcgcauauuacuugaucaaag

SEQ ID NO: 32 - RNA, 83 nt, Artificial
  3′ UTR sequence
cgcacaguggauccuaggcgauucuagaucacgcuguacguucacuucuucacugacucggaggaagugcaaacaaccccaaa

SEQ ID NO: 33 - RNA, 71 nt, Artificial
  IGR sequence
accagggccccugggcgcaccccccuccgggggugcgcccgggggcccccggccccauggggccgguuguu

SEQ ID NO: 34 - RNA, 75 nt, Artificial
  IGR sequence
gcccuagccucgacaugggccucgacgucacuccccaauaggggagugacgucgaggccucugaggacuugagcu

SEQ ID NO: 35 - DNA, 87 bp, Artificial
  hepatitis delta virus ribozyme
agctctcccttagccatccgagtggacgacgtcctccttcggatgcccaggtcggaccgcgaggaggtggagatgccatgccgaccc

SEQ ID NO: 36 - DNA, 36 bp, Artificial
  primer
cgatgctagcgccaccatggccgctctaactccgtc

SEQ ID NO: 37 - DNA, 57 bp, Artificial
  primer
atcgctcgagttacttgtcgtcatcgtctttgtagtcggtgtcgatgcccgtacgca

SEQ ID NO: 38 - DNA, 41 bp, Artificial
  primer
cgatgctagcgccaccatggaggtacgtgtgccaaactttc

SEQ ID NO: 39 - DNA, 32 bp, Artificial
  primer
atcgctcgagttaagcgtaatctggaacatcg

SEQ ID NO: 40 - DNA, 32 bp, Artificial
  primer
tatgggtaccaaccacactccataaaagtcag

(19) United States
(12) Patent Application Publication
(10) Pub. No.: US 2023/0310574 A1
(43) Pub. Date: Oct. 5, 2023

(54) MULTIVALENT VACCINES AGAINST TURKEY ARTHRITIS REOVIRUS

(71) Applicant: REGENTS OF THE UNIVERSITY OF MINNESOTA, Minneapolis, MN (US)

(72) Inventors: Sunil Kumar Mor, Minneapolis, MN (US); Yuying Liang, Minneapolis, MN (US); Hinh Ly, Minneapolis, MN (US); Sagar Mal Goyal, Minneapolis, MN (US); Robert E. Porter, Jr., Minneapolis, MN (US); Pawan Kumar, Minneapolis, MN (US)

(21) Appl. No.: 18/019,547

(22) PCT Filed: Aug. 5, 2021
(86) PCT No.: PCT/US2021/044614

§ 371 Date: Feb. 3, 2023

Related U.S. Application Data
(60) Provisional application No. 63/061,955, filed on Aug. 6, 2020.

(51) Int. Cl. A61K 39/145; A61P 31/14; C12N 15/86

(52) U.S. Cl. CPC A61K 39/145; A61P 31/14; C12N 15/86; A61K 2039/552

(57) ABSTRACT

Provided herein are genetically engineered Pichinde viruses that include three ambiense genomic segments. The first genomic segment includes two coding regions encoding a Z protein and a L RdRp protein. The second genomic segment includes a coding region encoding a nucleoprotein (NP) and the third genomic segment includes a coding region encoding a glycoprotein. Each of the second and third genomic segments include an additional coding region that may encode a reovirus sigma C protein or a reovims sigma B protein. In one embodiment, a genetically engineered Pichinde virus encodes a reovims sigma C protein and a reovirus sigma B protein. Also provided herein is a reverse genetics system for making genetically engineered Pichinde virus, and a collection of vectors that can be used to produce genetically engineered Pichinde virus. Further provided are methods for using a reverse genetics system, and methods for treating a reovirus infection in a subject.

### CROSS-REFERENCE TO RELATED APPLICATIONS

This application claims the benefit of U.S. Provisional Application Ser. No. 63/061,955, filed Aug. 6, 2020, which is incorporated by reference herein in its entirety.

### SUMMARY OF THE APPLICATION

Vaccination may be an effective way to reduce turkey arthritis reovirus infection in turkey flocks; however, there are currently no commercial vaccines available against turkey arthritis reovirus infection. Described herein is the use of reverse genetics technology to generate a recombinant Pichinde virus that expresses either the sigma C protein, the sigma B protein, or the combination thereof, as antigens.

Terms used herein will be understood to take on their ordinary meaning in the relevant art unless specified otherwise. Several terms used herein and their meanings are set forth below.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. Coding sequence, non-coding sequence, and regulatory sequence are defined below. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, “genetically modified” and “genetically engineered” refers to a Pichinde virus, which has been modified and is not found in any natural setting. For example, a genetically modified Pichinde virus is one into which has been introduced an exogenous polynucleotide, such as a coding region not typically present in a Pichinde virus. Another example of a genetically modified Pichinde virus is one, which has been modified to include three genomic segments.

A “coding region” is a nucleotide sequence that encodes an RNA molecule. The boundaries of a coding region are generally determined by a transcription initiation site at its 5′ end and a transcription terminator at its 3′ end. A coding region typically includes at least one nucleotide sequence that encodes a protein. A nucleotide sequence encoding a protein, also referred to as an open reading frame (ORF), has boundaries that are generally determined by a translation start codon at its 5′ end and a translation stop codon at its 3′ end. A coding region can encode an RNA molecule that includes two or more open reading frames. An RNA molecule that includes two or more open reading frames is referred to as a “polycistronic message.”

As used herein, the term “protein” refers broadly to a polymer of two or more amino acids joined together by peptide bonds. The term “protein” also includes molecules, which contain more than one protein joined by disulfide bonds, ionic bonds, or hydrophobic interactions, or complexes of proteins that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long-term culture in tissue culture medium). “In vivo” refers to cells that are within the body of a subject.

While the polynucleotide sequences described herein are listed as DNA or RNA sequences, it is understood that the complements, reverse sequences, and reverse complements of the DNA and RNA sequences can be easily determined by the skilled person. It is also understood that the sequences disclosed herein as DNA sequences can be converted from a DNA sequence to an RNA sequence by replacing each thymidine nucleotide with a uridine nucleotide. Likewise, the sequences disclosed herein as RNA sequences can be converted from a RNA sequence to a DNA sequence by replacing each uridine nucleotide with a thymidine nucleotide.

The term “and/or” means one or all of the listed elements or a combination of any two or more of the listed elements.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

For any method disclosed herein that includes discrete steps, the steps may be conducted in any feasible order. And, as appropriate, any combination of two or more steps may be conducted simultaneously.

### BRIEF DESCRIPTION OF THE FIGURES

The following detailed description of illustrative embodiments of the present disclosure may be best understood when read in conjunction with the following drawings.

FIG. 1A-B show the amino acid sequences of a sigma C (SEQ ID NO:5, FIG. 1A) and a sigma B (SEQ ID NO:7, FIG. 1B) protein and an example of a polynucleotide encoding each. The two polynucleotide sequences are codon optimized for expression in a mammalian cell.

FIG. 2A-2B show the alignment of 19 avian reovirus sigma B proteins. The location of amino acids that are identical to the top sequence are shown as a dot. A consensus sequence is also shown, as well as a depiction of the level of conservation at each residue. The level of conservation at each position is shown as a range from 0% to 100%. MVDL_SKM121_SB_Turkey, SEQ ID NO:9; AAR27797.1_TX98_Turkey, SEQ ID NO:10; AJW82019.1_Turkey, SEQ ID NO:11; AAR27796.1_TX99_Turkey, SEQ ID NO:12; AAM10637.2_NC98_Turkey, SEQ ID NO:13; ALG03384.1_D1246_Turkey, SEQ ID NO:14; ALG03372.1_D1104_Turkey, SEQ ID NO:15; AAF91191.1_601SI_Chicken, SEQ ID NO:16; ASG92570.1_DVB04_Chicken, SEQ ID NO:17; AAB61604.1_1733_Chicken, SEQ ID NO:18; AIS22878.1_GuangxiR2_ Chicken, SEQ ID NO:19; AKH03073.1_ HB10-1_ Chicken, SEQ ID NO:20; AKH03085.1_ LN09-1_ Chicken, SEQ ID NO:21; AWK97792.1_ MS01_ Layerchicken, SEQ ID NO:22; AIS22939.1_S1133_Chicken, SEQ ID NO:23; AIS22890.1_C78_Chicken, SEQ ID NO:24; YP 004226529.1_ AVS-B_Chicken, SEQ ID NO:25; ABA43655.1_Muscovy_duck, SEQ ID NO:26; AHL21601.1_Goose, SEQ ID NO:27; and consensus, SEQ ID NO:28.

FIG. 3 shows an electrophoretic gel picture of reovirus genes and PICV plasmids: A) RT-PCR amplification of full length 51 (GC) and S3 (GB) open reading frames of turkey arthritis reovirus: Lanes 1, 2, 3: S1 gene (1031 bp); M: Marker; Lanes 4, 5, 6: S3 gene (1157 bp); B) Restriction double digestion confirms the presence of 51 and S3 genes of TARV in recombinant PICV plasmids.

FIG. 4A-4B show GFP expression in BSRT-7-5 and BHK-21 cells: FIG. 4A) BSRT7-5 cells indicating successful rescue of viable recombinant PICV vaccine virus following transfection. FIG. 4B) GFP expression in BHK-21 cells infected with transfection supernatant after 96 hours post infection.

FIG. 5A-5F shows fluorescence in BHK-21 cells (Green fluorescent cells) after direct fluorescent antibody indicates TARV protein expression: FIG. 5A) SKM121 wild virus control; FIG. 5B) PICV-Bivalent SKM121 codon optimized; FIG. 5C) PICV-Bivalent SKM121 wild type; FIG. 5D) PICV-Monovalent SKM121 wild type; FIG. 5E) PICV-Monovalent SKM121 codon optimized; FIG. 5F) Negative control.

FIG. 6 shows SN antibody titers of individual birds of different groups at different ages at 3- and 5-weeks of age in different groups: MonovalentS1: Monovalent PICV-SKM121 S1 (codon-optimized) recombinant vaccine; MonovalentS3: Monovalent PICV-SKM121 S3 (codon-optimized) recombinant vaccine; Bivalent S1/S3: Bivalent PICV-SKM121 S1/S3 (codon-optimized) recombinant vaccine; Vaccine Control: PICV vaccine with no insert (control). A281 to A300 represent tags of each individual bird. The dotted lines represent the mean values at 3-week-old and the solid lines represent the mean values at 5-week-old. The values of Monovalent S3 and Bivalent S1/S3 groups were significantly higher than the other two groups at 3-week-old (plus sign) and values of Bivalent S1/S3 group were significantly higher than the other three groups at 5-week-old (asterisk sign) using the Mann-Whitney U test at P<0.05.

FIG. 7 shows serum neutralization (SN) antibody titers against TARV-SKM121. At 14 days of age (5 days after booster vaccination), the vaccinated poults and sentinels had significantly higher mean SN antibody titers to SKM121 than non-vaccinated poults. Box plots with different alphabets have significant difference between means at p<0.05 (non-parametric Kruskal Wallis test followed by pairwise Wilcoxon rank sum test).

FIG. 8 shows body weight at 35 days of age. Box plots with different letters have significant difference at p<0.05.

FIG. 9A-9B show reovirus gene copy numbers at 35 days of age in FIG. 9A) Intestine and FIG. 9B) Gastrocnemius tendon. Box plots with different letters have a significant difference between means at p<0.05 (non-parametric Kruskal Wallis test followed by pairwise Wilcoxon rank sum test).

FIG. 10 shows histologic lesion scores in poult gastrocnemius tendons at 35 days of age. Box plots with different letters have significant difference between means at p<0.05 (non-parametric Kruskal Wallis test followed by pairwise Wilcoxon rank sum test).

### DETAILED DESCRIPTION

Provided herein is a reverse genetics system for producing genetically modified Pichinde virus that expresses one or more proteins of avian reovirus. The genetically modified Pichinde virus-based reverse genetics system described herein has multiple advantages over other arenavirus systems for expression of avian reovirus proteins and use in immunizing subjects. Pichinde virus is not known to cause disease in humans, and there is evidence that Pichinde virus can cause asymptomatic human infections in a laboratory setting. For instance, 46% of laboratory personnel working with the virus are serum positive but do not show a distinct illness (Buchmeier et al., 2007, Arenaviridae: the viruses and their replication. In: Knipe and Howley (eds), Fields Virology. 5th ed. Philadelphia, PA: Lippincott Williams & Wilkins. pp. 1791-1827). The modified Pichinde virus described herein is further attenuated in comparison to the parental virus used in the human-infection study reported by Buchmeier et al. The modified Pichinde virus is genetically stable through serial passages in cell cultures. General human populations and animals (except for rice rats in Colombia, South America, where the virus was originally discovered) are not known to have prior exposure to Pichinde virus, which makes it an ideal vector for vaccine development due to the lack of pre-existing immunity against this Pichinde virus vector.

The reverse genetics system for this modified Pichinde virus includes three genomic segments. The first genomic segment includes two coding regions, one that encodes a Z protein and a second that encodes a RNA-dependent RNA polymerase (L RdRp). The second genomic segment includes a coding region that encodes a nucleoprotein (NP), and one or more additional coding regions that encode one or more avian reovirus proteins. The third genomic segment includes a coding region that encodes a glycoprotein, and one or more additional coding regions that encode one or more avian reovirus proteins. The second and third genomic segments can encode the same or different avian reovirus proteins.

The Z protein, L RdRp, NP protein, and glycoprotein are those encoded by a Pichinde virus. The Z protein is a small RING-domain containing matrix protein that mediates virus budding and regulates viral RNA synthesis. One example of a Z protein from a Pichinde virus is the sequence available at GenBank accession number ABU39910.1 (SEQ ID NO:1). The L RdRp protein is a RNA-dependent RNA polymerase that is required for viral DNA synthesis. One example of a L RdRp protein from a Pichinde virus is the sequence available at GenBank accession number ABU39911.1 (SEQ ID NO:2). The NP protein encapsidates viral genomic RNAs, is required for viral RNA synthesis, and suppresses host innate immune responses. One example of an NP protein from a Pichinde virus is the sequence available at GenBank accession number ABU39909.1 (SEQ ID NO:3). The glycoprotein is post-translationally processed into a stable signal peptide (SSP), the receptor-binding G1 protein, and the transmembrane G2 protein. One example of a glycoprotein from a Pichinde virus is the sequence available at GenBank accession number ABU39908.1 (SEQ ID NO:4).

Other examples of Z proteins, L RdRp proteins, NP proteins, and glycoprotein include proteins having structural similarity with a protein that is encoded by a Pichinde virus, for instance, SEQ ID NO:1, 2, 3, and/or 4. Structural similarity of two proteins can be determined by aligning the residues of the two proteins (for example, a candidate protein and a reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein, such as SEQ ID NO:1, 2, 3, or 4. A candidate protein is the protein being compared to the reference protein. A candidate protein may be isolated, for example, from a cell of an animal, such as a mouse, or can be produced using recombinant techniques, or chemically or enzymatically synthesized. A candidate protein may be inferred from a nucleotide sequence present in the genome of a Pichinde virus.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general parameters: expect threshold=10, word size=3, short queries=on; scoring parameters: matrix=BLOSUM62, gap costs=existence:11 extension:1, compositional adjustments=conditional compositional score matrix adjustment. Alternatively, proteins may be compared using the BESTFIT algorithm in the GCG package (version 10.2, Madison WI).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

The skilled person will recognize that the Z protein depicted at SEQ ID NO:1 can be compared to Z proteins from other arenaviruses, including Lassa virus (073557.4), LCMV Armstrong (AAX49343.1), and Junin virus (NP 899216.1) using readily available algorithms such as ClustalW to identify conserved regions of Z proteins. ClustalW is a multiple sequence alignment program for nucleic acids or proteins that produces biologically meaningful multiple sequence alignments of different sequences (Larkin et al., 2007, ClustalW and ClustalX version 2, Bioinformatics, 23(21):2947-2948). Using this information the skilled person will be able to readily predict, with a reasonable expectation that certain conservative substitutions to a Z protein such as SEQ ID NO:1 will not decrease activity of the protein.

The skilled person will recognize that the L RdRp protein depicted at SEQ ID NO:2 can be compared to L RdRp proteins from other arenaviruses, including Lassa virus (AAT49002.1), LCMV Armstrong (AAX49344.1), and Junin virus (NP 899217.1) using readily available algorithms such as ClustalW to identify conserved regions of L RdRp proteins. Using this information the skilled person will be able to readily predict with a reasonable expectation that certain conservative substitutions to an L RdRp protein such as SEQ ID NO:2 will not decrease activity of the protein.

The skilled person will recognize that the NP protein depicted at SEQ ID NO:3 can be compared to NP proteins from other arenaviruses, including Lassa virus (P13699.1), LCMV Armstrong (AAX49342.1), and Junin virus (NP 899219.1) using readily available algorithms such as ClustalW to identify conserved regions of NP proteins. Using this information the skilled person will be able to readily predict with a reasonable expectation that certain conservative substitutions to a NP protein such as SEQ ID NO:3 will not decrease activity of the protein.

The skilled person will recognize that the glycoprotein depicted at SEQ ID NO:4 can be compared to glycoproteins from other arenaviruses, including Lassa virus (P08669), LCMV Armstrong (AAX49341.1), and Junin virus (NP 899218.1) using readily available algorithms such as ClustalW to identify conserved regions of glycoproteins. Using this information the skilled person will be able to readily predict with a reasonable expectation that certain conservative substitutions to a glycoprotein such as SEQ ID NO:4 will not decrease activity of the protein.

Thus, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence. Alternatively, as used herein, a Pichinde virus Z protein, L RdRp protein, an NP protein, or a glycoprotein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence. Unless noted otherwise, “Z protein,” “L RdRp protein,” “NP protein,” and “glycoprotein” refer to a protein having at least 80% amino acid identity to SEQ ID NO:1, SEQ ID NO:2, SEQ ID NO:3, and SEQ ID NO:4, respectively.

A Z protein, L RdRp protein, an NP protein, or a glycoprotein having structural similarity the amino acid sequence of SEQ ID NO:1, 2, 3, or 4, respectively, has biological activity. As used herein, “biological activity” refers to the activity of Z protein, L RdRp protein, an NP protein, or a glycoprotein in producing an infectious virus particle. The biological role each of these proteins play in the biogenesis of an infectious virus particle is known, as are assays for measuring biological activity of each protein.

In one embodiment, the NP protein may include one or more mutations. A mutation in the NP protein may result in a NP protein that continues to function in the production of infectious viral particles, but has a decreased ability to suppress the production of certain cytokines by a cell infected with a Pichinde virus. A Pichinde virus that has decreased ability to suppress cytokine production is expected to be useful in enhancing an immunological response to a protein encoded by the virus. Examples of mutations include the aspartic acid at residue 380, the glutamic acid at residue 382, the aspartic acid at residue 457, the aspartic acid at residue 525, and the histidine at residue 520. A person of ordinary skill in the art recognizes that the precise location of these mutations can vary between different NP proteins depending upon the presence of small insertions or deletions in the NP protein, thus the precise location of a mutation is approximate, and can vary by 1, 2, 3, 4, 5, or more amino acids.

In one embodiment, the mutation in the NP protein may be the replacement of the aspartic acid, glutamic acid, or histidine at residues 380, 382, 457, 525, and/or 520 with any other amino acid. In one embodiment, the mutation may be the conservative substitution of the aspartic acid, glutamic acid, or histidine at residues 380, 382, 457, 525, and/or 520. In one embodiment, the mutation may be the replacement of the aspartic acid, glutamic acid, or histidine at residues 380, 382, 457, 525, and/or 520 with a glycine or an alanine. In one embodiment, the NP protein may include a mutation at one, two, three, or four of the residues 380, 382, 457, 525, or 520, and in one embodiment the NP protein may include a mutation at all five residues.

In one embodiment, the glycoprotein may include one or more mutations. A mutation in the glycoprotein may result in a glycoprotein that impairs virus spreading in vivo. Examples of mutations include the asparagine at residue 20, and/or the asparagine at residue 404. A person of ordinary skill in the art recognizes that the precise location of these mutations can vary between different glycoproteins depending upon the presence of small insertions or deletions in the glycoprotein, thus the precise location of a mutation is approximate, and can vary by 1, 2, 3, 4, or 5 amino acids.

In one embodiment, the mutation in the glycoprotein may be the replacement of the asparagine residue 20 and/or 404 with any other amino acid. In one embodiment, the mutation may be the conservative substitution of the asparagine residue 20 and/or 404. In one embodiment, the mutation may be the replacement of the asparagine residue 20 and/or 404 with a glycine or an alanine.

In one embodiment, the second genomic segment and/or the third genomic segment each independently include one or more additional coding regions that include an open reading frame encoding one or more avian reovirus proteins.

In one embodiment, the avian reovirus protein is an avian reovirus sigma C protein, also referred to herein as reovirus sigma C protein, sigma C protein, and S1 protein. A sigma C protein is encoded by the polycistronic S1 genomic segment of reovirus. An example of a sigma C protein is the amino acid sequence at SEQ ID NO:5. Avian reovirus sigma C proteins include conserved domains, and examples of conserved domains are disclosed by Pitcovski and Goldenberg (WO 2009/093251, see Table 3).

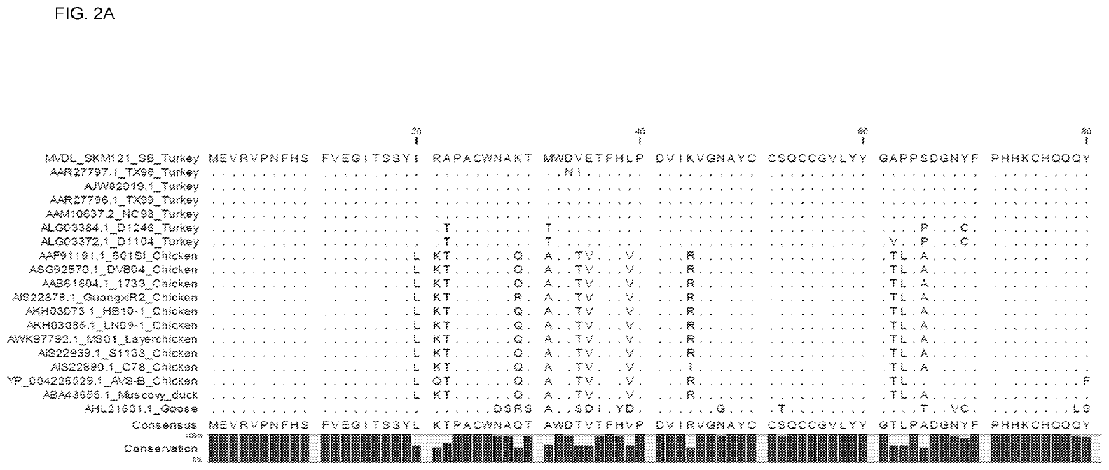

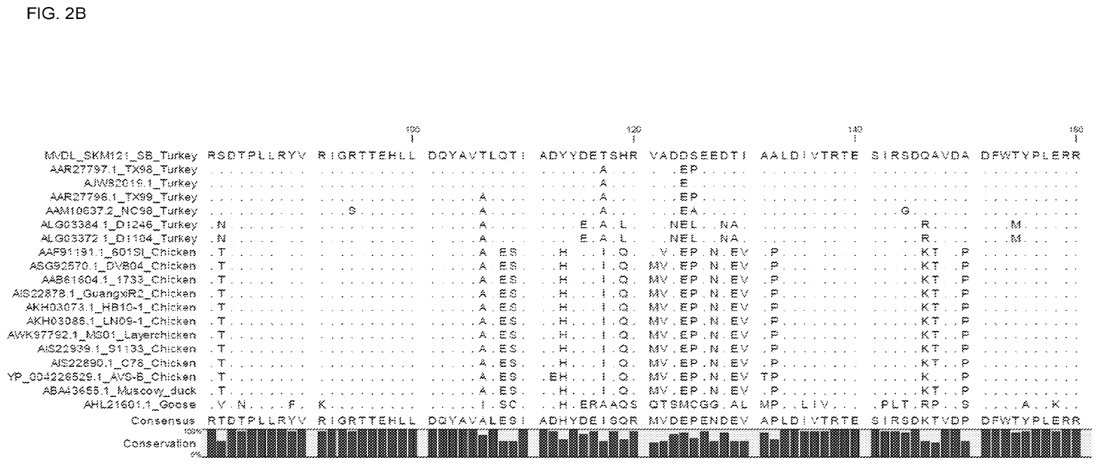

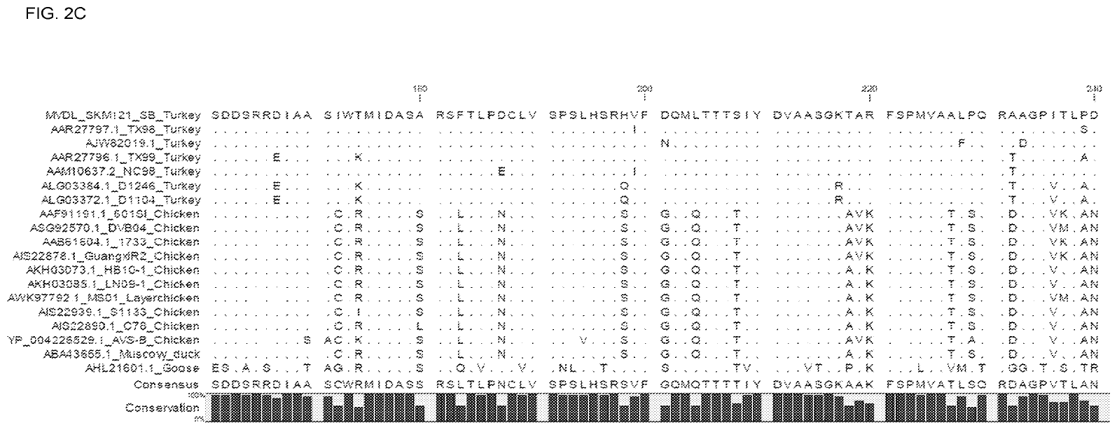

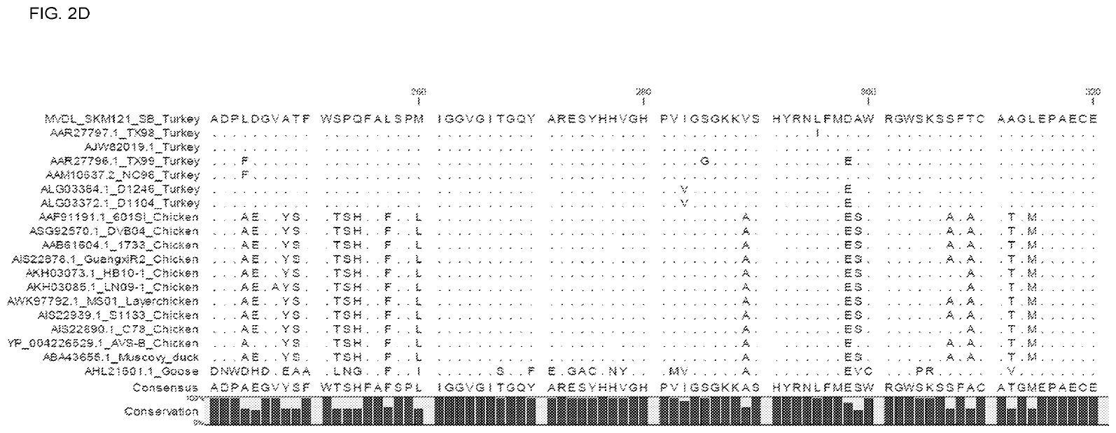

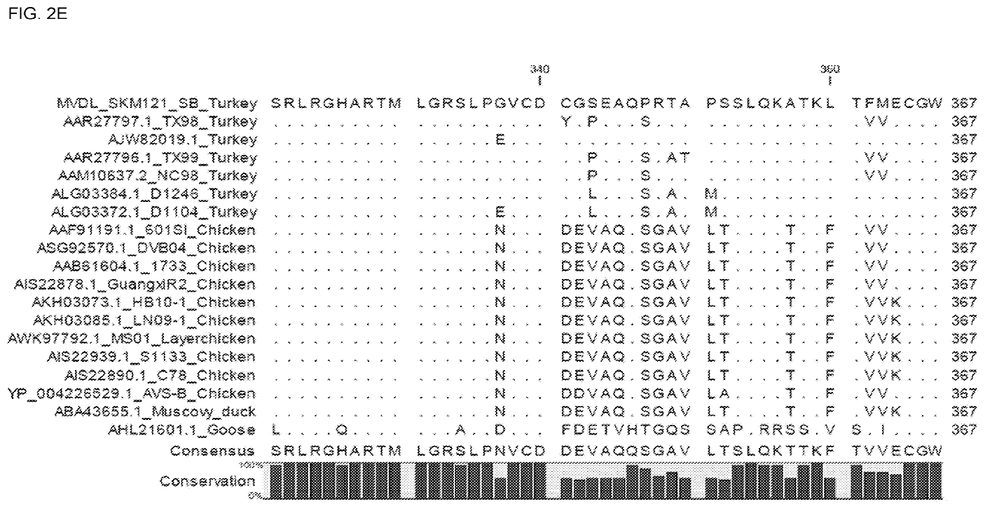

In one embodiment, the avian reovirus protein is an avian reovirus sigma B protein, also referred to herein as reovirus sigma B protein, sigma B protein, and S3 protein. A sigma B protein is encoded by the monocistronic S3 genomic segment of reovirus. An example of a sigma B protein is the amino acid sequence at SEQ ID NO:7. FIG. 2 shows an alignment of avian reovirus sigma B proteins and the location of amino acids that are identical to the top sequence are shown as a dot.

Other examples of sigma C proteins and sigma B proteins include proteins having structural similarity with a protein that is encoded by an avian reovirus, for instance, SEQ ID NO:5 or 7. Structural similarity of two proteins can be determined by aligning the residues of the two proteins (for example, a candidate protein and a reference sigma C or sigma B protein, such as SEQ ID NO:5 or 7) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate protein may be isolated, for example, from a reovirus, or can be produced using recombinant techniques, or chemically or enzymatically synthesized. A candidate protein may be inferred from a nucleotide sequence present in the genome of an avian reovirus.

Thus, as used herein, a sigma C protein or a sigma B protein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence, e.g., SEQ ID NO:5 or SEQ ID NO:7. Alternatively, as used herein, a sigma C protein or sigma B protein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence, e.g., SEQ ID NO:5 or SEQ ID NO:7. Unless noted otherwise, “sigma C protein” and “sigma B protein” refer to a protein having at least 80% amino acid identity to SEQ ID NO:5 or SEQ ID NO:7, respectively.

The skilled person will recognize that modifications to a sigma C protein and a sigma B protein within conserved domains are less desirable. Accordingly, in one embodiment substitutions of a sigma C protein and a sigma B protein can be present in regions that are not conserved.

An avian reovirus protein useful herein results in a humoral immune response, a cell-mediated immune response, or a combination thereof when expressed in a subject. In one embodiment, the protein is at least 6 amino acids in length. Any avian reovirus protein that results in a humoral immune response, a cell-mediated immune response, or a combination thereof when expressed in a subject can be used.

Pichinde virus is an arenavirus, and one characteristic of an arenavirus is an ambisense genome. As used herein, “ambisense” refers to a genomic segment having both positive sense and negative sense portions and coding strategies. For example, the first genomic segment of a Pichinde virus described herein is ambisense, encoding a Z protein in the positive sense and encoding a L RdRp protein in the negative sense. Thus, one of the two coding regions of the first genomic segment is in a positive-sense orientation and the other is in a negative-sense orientation. When the second and/or the third genomic segment includes a second coding region encoding a protein, the coding region encoding the protein is in a negative-sense orientation compared to the NP protein of the second genomic segment and to the glycoprotein of the third genomic segment.

Each genomic segment also includes nucleotides encoding a 5′ untranslated region (UTR) and a 3′ UTR. These UTRs are located at the ends of each genomic segment. Nucleotides useful as 5′ UTRs and 3′ UTRs include those present in Pichinde virus and are readily available to the skilled person (see, for instance, Buchmeier et al., 2007, Arenaviridae: the viruses and their replication. In: Knipe and Howley (eds), Fields Virology. 5th ed. Philadelphia, PA: Lippincott Williams & Wilkins. pp. 1791-1827). In one embodiment, a genomic segment that encodes a Z protein and an L RdRp protein includes a 5′ UTR sequence that is 5′ CGCACCGGGGAUCCUAGGCAUCUUUGGGUCACGCUUCAAAUUUGUCCAAUUUGAA CCCAGCUCAAGUCCUGGUCAAAACUUGGG (SEQ ID NO:29) and a 3′ UTR sequence that is CGCACCGAGGAUCCUAGGCAUUUCUUGAUC (SEQ ID NO:30). In one embodiment, a genomic segment that encodes a NP protein or a glycoprotein includes a 5′ UTR sequence that is 5′ CGCACCGGGGAUCCUAGGCAUACCUUGGACGCGCAUAUUACUUGAUCAAAG (SEQ ID NO:31) and a 3′ UTR sequence that is 5′ CGCACAGUGGAUCCUAGGCGAUUCUAGAUCACGCUGUACGUUCACUUCUUCACUG ACUCGGAGGAAGUGCAAACAACCCCAAA (SEQ ID NO:32). Alterations in these sequences are permitted, and the terminal 27-30 nucleotides are highly conserved between the genomic segments.

Each genomic segment also includes an intergenic region (IGR) located between the coding region encoding a Z protein and the coding region encoding a L RdRp protein, between the coding region encoding a nucleoprotein and additional coding region, and between the coding region encoding a glycoprotein and additional coding region. Nucleotides useful as an intergenic region are those present in Pichinde virus and are readily available to the skilled person. In one embodiment, an IGR sequence of a genomic segment that encodes a Z protein and an L RdRp protein includes 5′ ACCAGGGCCCCUGGGCGCACCCCCCUCCGGGGGUGCGCCCGGGGGCCCCCGGCCCC AUGGGGCCGGUUGUU (SEQ ID NO:33). In one embodiment, an IGR sequence of a genomic segment that encodes a NP protein or a glycoprotein includes 5′ GCCCUAGCCUCGACAUGGGCCUCGACGUCACUCCCCAAUAGGGGAGUGACGUCGA GGCCUCUGAGGACUUGAGCU (SEQ ID NO:34).

In one embodiment, a coding region can include nucleotides that encode a protein that is useful as a detectable marker, e.g., a molecule that is easily detected by various methods. Examples include fluorescent proteins (e.g., green, yellow, blue, or red fluorescent proteins), luciferase, chloramphenicol acetyl transferase, and other molecules (such as c-myc, flag, 6×his, HisGln (HQ) metal-binding peptide, and V5 epitope) detectable by their fluorescence, enzymatic activity or immunological properties.

Polynucleotides

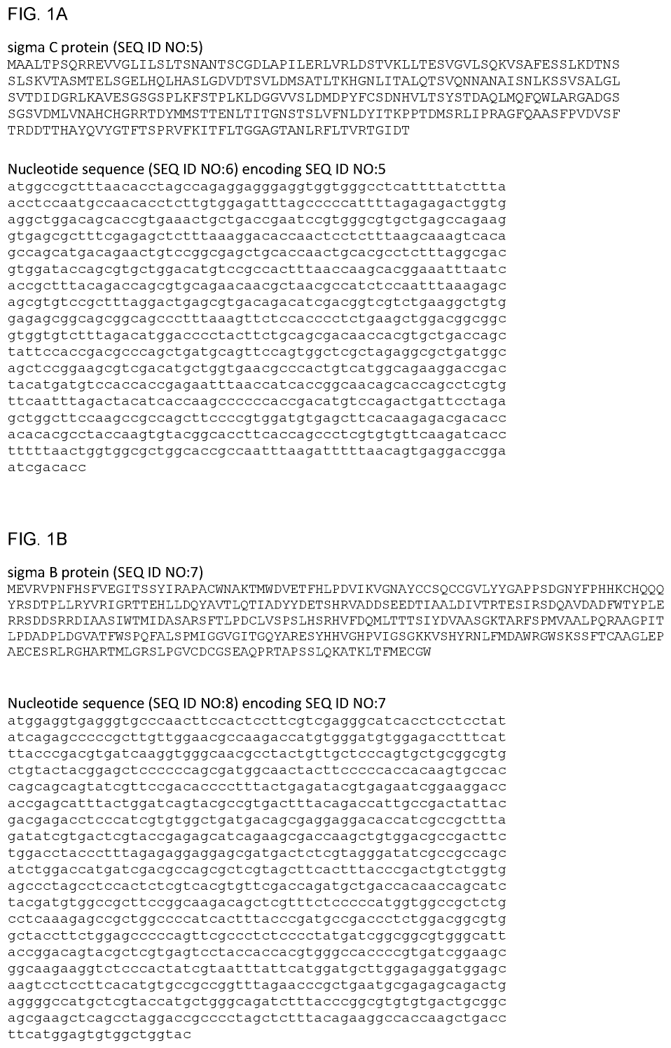

The present disclosure provides polynucleotides that encode any of the proteins described herein. Given the amino acid sequence of any one of the proteins described herein, a person of ordinary skill in the art can determine the full scope of polynucleotides that encode that amino acid sequence using conventional, routine methods. The nucleotide sequence encoding an avian reovirus protein can be modified to reflect the codon usage bias of a cell in which the protein will be expressed. The usage bias of nearly all cells in which a Pichinde virus would be expressed is known to the skilled person. For example, the open reading frames shown in FIG. 1 (SEQ ID NO:6, which encodes a sigma C protein, and SEQ ID NO:8, which encodes a sigma B protein) are optimized for expression in a mammalian cell.

Vectors

One or more of the genomic segments described herein can be present in a vector. For instance, all genomic segments can be present in one vector, two can be present in one vector, or each genomic segment is present in different vectors. In one embodiment, the sequence of a genomic segment in the vector is antigenomic, and in one embodiment the sequence of a genomic segment in the vector is genomic. As used herein, “anti-genomic” refers to a genomic segment that encodes a protein in the orientation opposite to the viral genome. For example, Pichinde virus is a negative-sense RNA virus. However, each genomic segment is ambisense, encoding proteins in both the positive-sense and negative-sense orientations. “Anti-genomic” refers to the positive-sense orientation, while “genomic” refers to the negative-sense orientation.

A vector is a replicating polynucleotide, such as a plasmid, phage, or cosmid, to which another polynucleotide may be attached so as to bring about the replication of the attached polynucleotide. Construction of vectors containing a genomic segment, and construction of genomic segments including insertion of a polynucleotide encoding a protein, employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of an RNA encoded by the genomic segment, i.e., an expression vector. The term vector includes, but is not limited to, plasmid vectors, viral vectors, cosmid vectors, or artificial chromosome vectors. Typically, a vector is capable of replication in a prokaryotic cell and/or a eukaryotic cell. In one embodiment, the vector replicates in prokaryotic cells, and not in eukaryotic cells. In one embodiment, the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells.

An expression vector optionally includes regulatory sequences operably linked to the genomic segment. The term “operably linked” refers to a juxtaposition of components such that they are in a relationship permitting them to function in their intended manner. A regulatory sequence is “operably linked” to a genomic segment when it is joined in such a way that expression of the genomic segment is achieved under conditions compatible with the regulatory sequence. One regulatory sequence is a promoter, which acts as a regulatory signal that bind RNA polymerase to initiate transcription of the downstream (3′ direction) genomic segment. The promoter used can be a constitutive or an inducible promoter. The present disclosure is not limited by the use of any particular promoter, and a wide variety of promoters is known. In one embodiment, a T7 promoter is used. Another regulatory sequence is a transcription terminator located downstream of the genomic segment. Any transcription terminator that acts to stop transcription of the RNA polymerase that initiates transcription at the promoter may be used. In one embodiment, when the promoter is a T7 promoter, a T7 transcription terminator is also used. Another example of a regulatory sequence is a Kozak sequence. In one embodiment, a ribozyme is present to aid in processing an RNA molecule. A ribozyme may be present after the sequences encoding the genomic segment and before a transcription terminator. An example of a ribozyme is a hepatitis delta virus ribozyme. One example of a hepatitis delta virus ribozyme is 5′ AGCTCTCCCTTAGCCATCCGAGTGGACGACGTCCTCCTTCGGATGCCCAGGTCGGAC CGCGAGGAGGTGGAGATGCCATGCCGACCC (SEQ ID NO:35).

Transcription of a genomic segment present in a vector results in an RNA molecule. When each of the three genomic segments is present in a cell the coding regions of the genomic segments are expressed and viral particles that contain one copy of each of the genomic segments are produced. The three genomic segments of the reverse genetics system described herein are based on Pichinde virus, an arenavirus with a segmented genome of two single-stranded ambisense RNAs. While the ability of the reverse genetics system to replicate and produce infectious virus typically requires the presence of the ambisense RNAs in a cell, the genomic segments described herein also include the complement thereof (i.e., complementary RNA), and the corresponding DNA sequences of the three RNA sequences.

A polynucleotide used to transform a host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, and neomycin.

Compositions

Also provided are compositions including a viral particle described herein, or the three genomic segments described herein. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition described herein may be referred to as a vaccine. The term “vaccine” as used herein refers to a composition that, upon administration to an animal, will increase the likelihood the recipient mounts an immune response to a protein encoded by one of the genomic segments described herein.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the compound of the present disclosure may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, phosphate buffered saline (PBS), and the like. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a viral particle described herein) in the required amount in an appropriate solvent with one or a combination of ingredients enumerated above, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds can be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

A composition can also include an adjuvant. An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest. Adjuvants may include, for example, IL-1, IL-2, emulsifiers, muramyl dipeptides, dimethyl dioctadecyl ammonium bromide (DDA), avridine, aluminum hydroxide, alum, magnesium hydroxide, oils, saponins, alpha-tocopherol, polysaccharides, emulsified paraffins, ISA-70, RIBI, TLR agonists, and other substances known in the art. It is expected that proteins as described herein will have immunoregulatory activity and that such proteins may be used as adjuvants that directly act as T cell and/or B cell activators or act on specific cell types that enhance the synthesis of various cytokines or activate intracellular signaling pathways. Such proteins are expected to augment the immune response to increase the protective index of the existing composition.

The data obtained from cell culture assays and animal studies can be used in formulating a range of dosage for use in an animal. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

The compositions can be administered once to result in an immune response, or one or more additional times as a booster to potentiate the immune response and increase the likelihood immunity to the proteins is long-lasting. A composition of the present disclosure may be administered in an amount sufficient to treat certain conditions as described herein. The amount of protein or vector present in a composition as described herein can vary. In one embodiment, a dosage of viral particles or plaque forming units (PFU) can be at least 1×10⁴, at least 5×10⁴, at least 1×10⁵, at least 5×10⁵, at least 1×10⁶ viral particles, at least 5×10⁶ viral particles, at least 1×10′ viral particles, and no greater than 1×10⁹, no greater than 5×10⁸, no greater than 1 x 10⁸, no greater than 5×10′, no greater than 1×10′, or no greater than 5×10⁶ viral particles. In one embodiment, a dosage of viral particles or PFU can be at least 1×10⁴, to no greater than 1 x 10⁹. The skilled artisan will appreciate that certain factors may influence the dosage and timing required to effectively treat a subject, including but not limited to the severity of the disease or disorder, previous treatments, the general health and/or age of the subject, and other diseases present.

Methods of Use

Also provided herein are methods for using the genomic segments. In one embodiment, a method includes making an infectious viral particle. Such a method includes, but is not limited to, providing a cell that includes each of the three genomic segments described herein (a first genomic segment, a second genomic segment, and a third genomic segment) and incubating the cell under conditions suitable for generating full-length genomic RNA molecules of each genomic segment. The full-length genomic RNA of each genomic segment is antigenomic.

Production of full-length genomic RNA molecules of each genomic segment results in transcription and translation of each viral gene product and amplification of the viral genome to generate infectious progeny virus particles. As used herein, an “infectious virus particle” refers to a virus particle that can interact with a suitable eukaryotic cell, such as an avian or mammalian cell, to result in the introduction of the three genomic segments into the cell, and the transcription of the three genomic segments in the cell. The method can also include introducing into the cell vectors that encode the three genomic segments. Infectious virus particles are released into a supernatant and may be isolated and amplified further by culturing on a eukaryotic cell, such as, but not limited to, baby hamster kidney (BHK21) epithelial cells or African green monkey epithelial (VERO) cells. The method may include isolating a viral particle from a cell or a mixture of cells and cellular debris. The method may include inactivating virus particles using standard methods, such a hydrogen peroxide treatment. Also provided is a viral particle, infectious or inactivated, that contains three genomic segments described herein. In one embodiment, a composition can be defined by the number of viral particles present. In one embodiment, a composition can be defined by the number of plaque forming units (PFU) present. The number of PFU present can be determined by plating on a cell line such as Vero cells. In one embodiment, a viral particle is replication competent.

In one embodiment, a method includes expression of one or more avian reovirus proteins in a cell. Such a method includes, but is not limited to, introducing into a cell the three genomic segments described herein. In one embodiment, a virus particle that is infectious or inactivated is introduced into a cell. The second and/or the third genomic segment may include one or more additional coding regions encoding an avian reovirus protein. More than one type of virus particle may be administered. For instance, two populations of virus particles may be administered where each population encodes different proteins. The cell is a suitable eukaryotic cell, such as an avian cell. In one embodiment, the avian cell is a chicken embryonic fibroblast. In one embodiment, the avian cell is a turkey cell. The cell may be ex vivo or in vivo. The three genomic segments may be introduced by contacting a cell with an infectious virus particle that contains the three genomic segments, or by introducing into the cell vectors that include the genomic segments. The method further includes incubating the cell under conditions suitable for expression of the coding regions present on the three genomic segments.

In one embodiment, a method includes immunizing an animal. Such a method includes, but is not limited to, administering to an animal a viral particle that is infectious or inactivated, that contains the three genomic segments described herein. Optionally, the administration can be followed by one or more booster administrations. In one embodiment, a booster can be administered at least 1 week, at least 2 weeks, or at least 3 weeks after the first administration. The second and/or the third genomic segment may include a second coding region that encodes an antigen. The second and third genomic segments may encode the same antigen, e.g., a sigma C protein or a sigma B protein, or one may encode a sigma C protein and the other encode a sigma B protein. More than one type of virus particle may be administered. For instance, two populations of virus particles may be administered where each population encodes different antigens, e.g., one encodes sigma C proteins and another encodes sigma B proteins. Immunization will provide protection against reovirus expressing identical sigma C and/or sigma B proteins (e.g., homologous protection) as well as reovirus expressing sigma C and/or sigma B proteins having structural similarity to the sigma C and sigma B proteins described in the present disclosure (e.g., heterologous protection).

The animal may be any animal in need of immunization, including a vertebrate, such as an avian. The animal can be, for instance, poultry, including domesticated poultry such as a chicken or turkey. In one embodiment, the animal is an animal at risk of exposure to an avian reovirus. In another embodiment, the animal is an animal that has an avian reovirus infection. The immune response may be a humoral response (e.g., the immune response includes production of antibody in response to an antigen), a cellular response (e.g., the activation of phagocytes, antigen-specific cytotoxic T-lymphocytes, and the release of cytokines in response to an antigen), or a combination thereof.

In another embodiment, a method includes treating an avian reovirus infection in an animal. As used herein, the term “infection” refers to the presence of and multiplication of an avian reovirus in the body of a subject. The infection can be clinically inapparent or result in signs associated with disease caused by avian reovirus. The infection can be at an early stage, or at a late stage. As used herein, the term “disease” refers to any deviation from or interruption of the normal structure or function of a part, organ, or system, or combination thereof, of a subject that is manifested by a characteristic sign. In another embodiment, a method includes treating one or more signs of avian reovirus in an animal. In one embodiment, the method includes administering an effective amount of a composition described herein to an animal having or at risk of having a sign of avian reovirus infection, and optionally determining whether the amount of avian reovirus in the animal decreases.

Treatment of infection and/or sign associated with avian reovirus can be prophylactic or, alternatively, can be initiated after the development of an infection, symptom, and/or sign. As used herein, the term “sign” refers to objective evidence in a subject of a condition caused by infection by disease. Signs associated with avian reovirus and the evaluations of such signs are routine and known in the art. Examples of signs of avian reovirus include, but are not limited to, conditions such as viral arthritis/tenosynovitis and stunting syndrome. Viral arthritis/tenosynovitis includes swelling of one or both hock (tibiotarsal-tarsometatarsal) joints, the main load-bearing joint in a bird, causing acute lameness. Stunting syndrome is characterized by a considerably reduced live weight in affected birds and various degrees of non-uniformity in a flock, varying from 5-10% to 40-50% and typically seen after the age of 14 days. Treatment that is prophylactic, for instance, initiated before a subject manifests signs of avian reovirus infection, is referred to herein as treatment of a subject that is “at risk” of developing avian reovirus. Accordingly, administration of a composition can be performed before, during, or after the occurrence of avian reovirus infection. Treatment initiated after the development of an avian reovirus infection may result in decreasing the severity of the signs of a condition caused by avian reovirus, or completely removing the signs. In this aspect of the disclosure, an “effective amount” is an amount effective to prevent the manifestation of signs of a condition caused by avian reovirus, decrease the severity of the signs, and/or completely remove the signs.

Also provided herein is a kit for immunizing an animal. The kit includes viral particles as described herein, where the second and/or third genomic segments each independently include a coding region that encodes an antigen, in a suitable packaging material in an amount sufficient for at least one immunization. In one embodiment, the kit may include more than one type of viral particle, e.g., the kit may include one viral particle that encodes one or two antigens and a second viral particle that encodes one or two other antigens. Optionally, other reagents such as buffers and solutions needed to practice the present disclosure are also included. Instructions for use of the packaged viral particles are also typically included.

As used herein, the phrase “packaging material” refers to one or more physical structures used to house the contents of the kit. The packaging material is constructed by known methods, preferably to provide a sterile, contaminant-free environment. The packaging material has a label, which indicates that the viral particles can be used for immunizing an animal. In addition, the packaging material contains instructions indicating how the materials within the kit are employed to immunize an animal. As used herein, the term “package” refers to a solid matrix or material such as glass, plastic, paper, foil, and the like, capable of holding within fixed limits viral particles. Thus, for example, a package can be a glass vial used to contain an appropriate amount of viral particles. “Instructions for use” typically include a tangible expression describing the amount of viral particles, route of administration, and the like.

The invention is defined in the claims. However, below there is provided a non-exhaustive listing of non-limiting exemplary aspects. Any one or more of the features of these aspects may be combined with any one or more features of another example, embodiment, or aspect described herein.

Exemplary Aspects

Aspect 1. A genetically engineered Pichinde virus comprising: three ambisense genomic segments, wherein the first genomic segment comprises a coding region encoding a Z protein and a coding region encoding a L RNA-dependent RNA polymerase (L RdRp) protein, wherein the second genomic segment comprises a coding region encoding a nucleoprotein and a coding region, wherein the coding region encodes a first avian reovirus protein, wherein the third genomic segment comprises a coding region encoding a glycoprotein and a coding region, wherein the coding region encodes a second avian reovirus protein, and wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

Aspect 2. The virus of Aspect 1 wherein the nucleoprotein comprises at least one mutation that reduces the exoribonuclease activity of the nucleoprotein, wherein the mutation is selected from an aspartic acid at about amino acid 380, a glutamic acid at about amino acid 382, an aspartic acid at about amino acid 525, a histidine at about amino acid 520, and an aspartic acid at about amino acid 457, wherein the aspartic acid, glutamic acid, or histidine is substituted with any other amino acid.

Aspect 3. The virus of any one of Aspects 1-2 wherein the glycoprotein comprises at least one mutation that alters the activity of the glycoprotein, wherein the mutation is selected from an asparagine at about amino acid 20 and an asparagine at about amino acid 404, and wherein the asparagine is substituted with any other amino acid.

Aspect 4. An infectious virus particle comprising the three genomic segments of any one of Aspects 1-3.

Aspect 5. A composition comprising the isolated infectious virus particle of any one of Aspects 1-4.

Aspect 6. A collection of vectors comprising: a first vector encoding a first genomic segment comprising a coding region encoding a Z protein and a coding region encoding a L RdRp protein, wherein the first genomic segment is antigenomic, a second vector encoding a second genomic segment comprising a coding region encoding a nucleoprotein and a coding region encoding a first avian reovirus protein, wherein the second genomic segment is antigenomic, and a third vector encoding a third genomic segment comprises a coding region encoding a glycoprotein and a coding region, wherein the coding region encodes a second avian reovirus protein, wherein the third genomic segment is antigenomic, wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

Aspect 7. The collection of Aspect 6 wherein the vectors are plasmids.

Aspect 8. The collection of Aspect 6 or 7 wherein the plasmids further comprise a T7 promoter.

Aspect 9. A method for making a genetically engineered virus particle comprising: introducing into a cell the collection of vectors of any one of Aspects 1-6; and incubating the cells in a medium under conditions suitable for expression and packaging of the first, second, and third genomic segments into a virus particle.

Aspect 10. The method of Aspect 9 wherein the virus particle is an infectious virus particle.

Aspect 11. The method of Aspect 9 further comprising isolating the virus particle.

Aspect 12. The method of any one of Aspects 9-11 wherein the cells express a T7 polymerase.

Aspect 13. The isolated virus particle produced by the method of any one of Aspects 9-11.

Aspect 14. A composition comprising the isolated virus particle of any one of Aspects 9-11.

Aspect 15. A reverse genetics system for making a genetically engineered virus comprising three vectors, wherein a first vector encodes a first genomic segment comprising a coding region encoding a Z protein and a coding region encoding a L RdRp protein, wherein the first genomic segment is antigenomic, wherein the second vector encodes a second genomic segment comprising a coding region encoding a nucleoprotein and a coding region encoding a first avian reovirus protein, wherein the second genomic segment is antigenomic, wherein the third vector encodes a third genomic segment comprises a coding region encoding a glycoprotein and a second avian reovirus particle, wherein the third genomic segment is antigenomic, wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

Aspect 16. The reverse genetics system of Aspect 15 wherein each plasmid comprises a T7 promoter.

Aspect 17. A method for using a reverse genetics system, comprising: introducing into a cell the three vectors of genomic segments of any one of Aspects 1-16; and incubating the cell under conditions suitable for the transcription of the three genomic segments and expression of the coding regions of each genomic segment.

Aspect 18. The method of Aspect 17 wherein the cell produces virus particles, the method further comprising isolating virus particles produced by the cell, wherein each virus particle comprises the three genomic segments.

Aspect 19. The method of Aspects 17-18 wherein the virus particles are infectious.

Aspect 20. The method of any one of Aspects 17-19 wherein the introducing comprises transfecting a cell with the three genomic segments.

Aspect 21. The method of any one of Aspects 17-20 wherein the introducing comprises contacting the cell with a virus particle comprising the three genomic segments.

Aspect 22. The method of any one of Aspects 17-21 wherein the cell is ex vivo.

Aspect 23. The method of any one of Aspects 17-22 wherein the cell is a vertebrate cell.

Aspect 24. The method of any one of Aspects 17-23 wherein the vertebrate cell is an avian cell.

Aspect 25. The method any one of Aspects 17-24 wherein the avian cell is a chicken embryonic fibroblast.

Aspect 26. A method for treating a subject, comprising administering to a subject the infectious virus particle of any one of Aspects 1˜4 or 9-13 or the composition of any one of Aspects 1-5 or 9-14 to result in an immune response, wherein the subject is at risk of infection by a reovirus.

Aspect 27. The method of Aspect 26 wherein the subject is a vertebrate.

Aspect 28. The method of any one of Aspects 27-28 wherein the vertebrate is avian, such as a turkey or chicken.

Aspect 29. The method of any one of Aspects 26-28 wherein the immune response comprises a humoral immune response.

Aspect 30. The method of any one of Aspects 26-29 wherein the immune response comprises a cell-mediated immune response.

Aspect 31. The sigma C protein of any of Aspects 1-30 wherein the sigma C protein comprises an amino acid sequence of at least 80% identity with SEQ ID NO:5.

Aspect 32. The sigma B protein of any of Aspects 1-30 wherein the sigma B protein comprises an amino acid sequence of at least 80% identity with SEQ ID NO:7.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Development of Recombinant Pichinde Virus Vectored Vaccine Against Turkey Arthritis Reovirus

Abstract

Vaccination may be an effective way to reduce turkey arthritis reovirus (TARV) infection in turkey flocks; however, there are currently no commercial vaccines available against TARV infection. Here, we describe the use of reverse genetics technology to generate a recombinant Pichinde virus (PICV) that expresses the 51 (sigma C) and/or S3 (sigma B) proteins of TARV as antigens. Nine recombinant PICVs were developed carrying the wild type 51 and/or S3 genes from three different TARV strains. In addition, three recombinant PICVs were produced carrying codon optimized 51 and/or S3 genes of a single TARV strain. The 51 and S3 antigens were found to be expressed in virus-infected cells via reverse transcriptase-polymerase chain reaction (RT-PCR) and direct fluorescent antibody (FA) technique using FITC-conjugated anti-avian reovirus antibodies. Turkey poults inoculated with the recombinant PICV vaccine expressing the bivalent TARV 51 and S3 antigens developed high anti-TARV antibody titers indicating the immunogenicity (and safety) of this vaccine. Future in vivo challenge studies using a turkey reovirus infection model will determine the optimum dose and protective efficacy of this recombinant virus-vectored vaccine. This Example is also available as Kumar et al. Pathogens. 2021, 10(2):197, doi: 10.3390/pathogens10020197. PMID: 33668435.

1. Introduction

Turkey arthritis reoviruses (TARVs) re-emerged in 2011 in Minnesota and other states in the US [1-3]. The viral genome consists of 10 segments of dsRNA grouped into large (L1, L2, L3), medium (M1, M2, M3), and small (S1, S2, S3, S4) based on migration pattern on polyacrylamide gel electrophoresis [4,5]. The genome has 12 open reading frames (ORFs), which encode for eight structural and four non-structural proteins. The proteins encoded by L, M and S genes are lambda (λ), mu (m) and sigma (σ), respectively [5]. The S1 and S3 segments translate into σC (cell attachment) and σB (outer capsid) proteins, respectively. The σC protein possesses both type and broad-specific epitopes, while σB protein contains group-specific neutralizing epitope [6]. TARV-infected turkeys display clinical disease in the form of lameness, tenosynovitis, and arthritis resulting in huge economic losses mainly due to culling. No commercial vaccine is available to protect turkey flocks from the emerging TARV strains. Some turkey producers rely on the use of autogenous vaccines. However, the evolving nature of the virus to create new mutant strains poses a challenge to regularly update the vaccines. Additionally, there is no live vaccine available that can be used for priming before boosting breeder turkeys with injectable killed vaccines.

Using a live and safe vectored vaccine to deliver TARV antigens is a reasonable alternative to live reovirus vaccines. Recently, a Pichinde virus (PICV) vector was developed that safely and effectively delivered the model antigens, the influenza viral hemagglutinin (HA) and nucleoprotein (NP) [7]. This arenavirus was first isolated from its natural host Oryzomys albigularis (rice rats) in the Pichinde valley of Colombia, South America [8]. Arenaviruses are enveloped RNA viruses with a bisegmented genome and are known to target dendritic cells and macrophages at early stages of infection, making it a potentially powerful vaccine vector [9,10,11,12].

Using reverse genetics technique, a live recombinant PICV (strain 18) with a tri-segmented RNA genome (rP18tri) has been developed to carry and express up to two foreign genes. One of the foreign genes could be the green fluorescent protein (GFP) that can be used to mark virus-infected cells in cell culture. The rP18tri has been shown to be attenuated both in vitro and in vivo and has the ability to induce cell-mediated and humoral immune responses [7]. For example, mice immunized with recombinant PICV-hemagglutinin (PICV-HA), expressing the modeled influenza hemagglutinin protein, developed a strong humoral response against HA that afforded complete protection against a lethal avian influenza virus infection. Additionally, the immune responses, both humoral immune response against the modeled influenza HA and cell-mediated immune response against the modeled influenza NP, increased significantly after a booster dose of the same vaccine [7].

The present study was undertaken to develop recombinant PICV vaccines expressing one or both TARV antigenic S1 and S3 proteins. In addition, the vaccines were administered to turkey poults to determine vaccine safety and efficacy. This is a ‘proof of concept’ study for the development and recovery of recombinant PICV-TARV viruses and testing the safety and immunogenic properties of the turkey reovirus S1 and S3 protein(s) in vivo.

2. Materials and Methods

2.1 Cells and Viruses: QT-35 cells were grown in Dulbecco's modified Eagle's medium (DMEM) containing 10% fetal bovine serum (FBS) and 50 ug/ml penicillin-streptomycin. Baby hamster kidney (BHK-21) cells, BSRT7-5 cells (BHK-21 cells stably expressing T7 RNA polymerase), Vero cells, and LMH cells were grown in Eagle's minimal essential medium (MEM) (Sigma-Aldrich) that contained 10% FBS, 1 ug/ml Gentamicin, and 50 ug/ml penicillin-streptomycin. Media for BSRT7-5 cells was also supplemented with 1 μg/ml Gentamicin (Invitrogen-Life Technologies). Three strains of turkey arthritis reovirus (SKM73, SKM95 and SKM121) isolated in QT-35 cells from tendons of lame turkeys were used. These viruses were selected based on their pathogenicity and genomic characterization.

2.2 Pichinde virus plasmids: Three plasmids were used: (i) pP18S1-GPC/MCS encodes the glycoprotein GPC and a multiple-cloning-site (MCS) to clone the gene of interest; (ii) pP18S2-MCS/NP encodes the nucleoprotein NP and a MCS; (iii) and pP18L plasmid (the L plasmid) expresses the full-length antigenomic strand of the rP18L segment under the control of T7 promoter and does not contain any specific site to clone foreign genes [7]. These three plasmids were obtained from Dr. Ly's laboratory at the University of Minnesota, Saint Paul, MN, USA.

2.3 Preparation of vectors and gene inserts: Genomic RNA was isolated from three TARV isolates (SKM73, SKM95, and SKM121). Full-length open reading frame (ORF) of S1 and S3 genomic segments of these viruses were amplified. Additionally, the S1 and S3 ORF sequences of SKM121 were codon-optimized and commercially custom synthesized in a pUC vector. The cDNA was synthesized using random primers and SuperScript™ IV First-Strand Synthesis (Thermo Fisher Scientific, Catalog #18091200, Waltham, MA, USA) and was PCR amplified using specific cloning primers (Table 1) and Phusion® High-Fidelity PCR Master Mix with HF Buffer (NEB Catalog #M0531S, Ipswich, MA, USA). The NheI and Kozak sequences were added in the forward primer so that these features are included in the amplified product. Similarly, XhoI and sequence tags (FLAG tag in S1 and HA tag in S3 gene) were added in the reverse primer. The reaction conditions were: 98° C. for 30 sec (initial denaturation); 5 cycles of denaturation at 98° C. for 10 sec, annealing at 66° C. for 30 sec and extension at 72° C. for 1 min; 30 cycles of denaturation 98° C. for 10 sec, annealing at 72° C. for 30 sec and extension at 72° C. for 1 min; final extension at 72° C. for 7 min and 4° C. hold. The PCR amplified products (S1 and S3 genes of all three isolates) and the plasmids of the PICV (pP18S1-GPC/MCS and pP18S2-MCS/NP) were restriction digested (NheI and XhoI, NEB) and gel purified using QIAquick gel extraction kit (Qiagen Catalog #28704, Germantown, MD, USA). The codon-optimized versions of ORF were extracted from the pUC vector by restriction double digestion.

TABLE 1
List of primers used for full-length open
reading frame amplification of S1 and S3
segments of turkey arthritis reovirus.

| Serial | Name | Sequence | Bases | RE | Tag |
|---|---|---|---|---|---|
| 1 | TARV- | 5′CGATGCTAGCGCC | 36 | NheI | — |
| | S1 F | ACCATGGCCGCTCTA | | | |
| | | ACTCCGTC3′ | | | |
| | | (SEQ ID NO: 36) | | | |
| 2 | TARV- | 5′ATCGCTCGAGTTA | 57 | XhoI | FLAG |
| | S1 R | CTTGTCGTCATCGTC | | | |
| | | TTTGTAGTCGGTGTC | | | |
| | | GATGCCCGTACGCA3′ | | | |
| | | (SEQ ID NO: 37) | | | |
| 3 | TARV- | 5′CGATGCTAGCGCC | 41 | NheI | — |
| | S3 F | ACCATGGAGGTACGT | | | |
| | | GTGCCAAACTTTC3′ | | | |
| | | (SEQ ID NO: 38) | | | |
| 4 | TARV- | 5′ATCGCTCGAGTTA | 64 | XhoI | HA |
| | S3 R | AGCGTAATCTGGAAC | | | |
| | | ATCG | | | |
| | | (SEQ ID NO: 39) | | | |
| | | TATGGGTACCAACCA | | | |
| | | CACTCCATAAAAGTC | | | |
| | | AG3′ | | | |
| | | (SEQ ID NO: 40) | | | |

2.4 Cloning and Transfection: The S1 and S3 gene inserts were ligated in MCS region of plasmids pP18S2-MCS/NP and pP18S1-GPC/MCS, respectively, using 5 U/μL of T4 DNA ligase (ThermoFisher Scientific, Catalog #EL0011). The ligation reaction mix was used to transform competent bacterial cells (DH5a) followed by selection using ampicillin antibiotic. All plasmids (plasmid pP18L, pP18S1-GPC/GFP, pP18S2-GFP/NP, and recombinant plasmids pP18S1-GPC/S3 and pP18S2-S1/NP) were isolated by plasmid midi prep kit (Sigma-Aldrich). Recombinant plasmids were PCR-confirmed for reovirus genes and sequence-confirmed for correct orientation and reading frame. The recombinant plasmids were used to transfect BSRT7-5 cells in various combinations (Table 2) using Lipofectamine™ 3000 transfection reagent (ThermoFisher, Catalog #L3000008) following manufacturer's instruction with minor modifications. Briefly, BSRT7-5 cells were grown in 6-well plates to 80% confluency. Four hours before transfection, the cells were washed, and fresh antibiotic-free media was added. For transfection, 8 μl of P3000 reagent, 2 μg of L-plasmid and 1 μg each of S1 and S2 plasmids were diluted in 250 μl of Opti-MEM (Invitrogen-Life Technologies) and incubated for 15 min at room temperature. In another tube, 10 μl of lipofectamine was diluted in 250 μl of Opti-MEM. Both mixtures were combined followed by incubation at room temperature for 20 min to prepare DNA-Lipid complexes. The cells were transfected with the resultant mixture, and MEM was changed after 4 h to remove the toxic lipofectamine. After 48, 72 and 96 h post transfection, cell supernatants were collected and stored at −80° C. Different monovalent and bivalent vector viruses were generated as detailed in Table 2. The resultant viral recovery was confirmed by observing green fluorescence of GFP in inoculated cell culture. The rescued virus was then grown in BHK-21 cells and the GFP green fluorescence was observed. The presence of reovirus genes was confirmed by gene-specific PCR.

TABLE 2
Pichinde virus (PICV) recombinant plasmids
used to generate vectored viruses

| | | | Insert in plas- | Insert in plas- |
|---|---|---|---|---|
| | Insert | Recombinant | mid 1 pP18S1- | mid 2 pP18S2- |
| No. | origin | vector virus | GPC/MCS | MCS/NP |
| 1 | SKM73* | Monovalent | GFP^{a} | S1⁺ wild type |
| 2 | | Monovalent | S3⁺ wild type | GFP |
| 3 | | Bivalent | S3 wild type | S1 wild type |
| 4 | SKM95* | Monovalent | GFP | S1 wild type |
| 5 | | Monovalent | S3 wild type | GFP |
| 6 | | Bivalent | S3 wild type | S1 wild type |
| 7 | SKM121* | Monovalent | GFP | S1 wild type |
| 8 | | Monovalent | S3 wild type | GFP |
| 9 | | Bivalent | S3 wild type | S1 wild type |
| 10 | SKM121* | Monovalent | GFP | S1 codon |
| | | | | optimized |
| 11 | | Monovalent | S3 codon | GFP |
| | | | optimized | |
| 12 | | Bivalent | S3 codon | S1 codon |
| | | | optimized | optimized |
| 13 | | Control | GFP | GFP |

^{a}GFP = Green Fluorescence Protein;
⁺S1 and S3 are genes inserted in to PICV plasmids;
*SKM73, SKM95 and SKM121 are turkey arthritis reoviruses whose S1 and S3 genes were inserted into PICV plasmids.

2.5 Detection of reovirus antigenic proteins: The expression of GC and GB proteins by recombinant tri-segmented PICV vaccine viruses in BHK-21 cells was verified by direct fluorescence antibody (FA) assay. The BHK-21 cells were inoculated with the recombinant PICVs and at 96 h post infection (hpi), the cells were harvested, plated on 12-chamber slides and dried for 2 hours. Cells were then fixed in acetone for 2 hours followed by the addition of polyclonal FITC-conjugated anti-avian reovirus antibodies (National Veterinary Services Laboratory, Ames, IA, USA, Reagent #680-ADV). After incubation at 37° C. in a CO₂ incubator for 2 hours, counter staining was done using 0.1% Evan Blue biological stain (EBBS). The slides were then mounted and examined under a fluorescent microscope to observe FITC green fluorescence, which was indicative of avian reovirus protein expression by the vectored viruses.

2.6 Vaccination experiment: In a pilot experiment, four groups of turkey poults (5 birds/group) were inoculated with 0.2 mL of the following recombinant PICVs containing codon optimized gene segments of TARV-SKM121 (monovalent PICV-S1, monovalent PICV-S3, bivalent PICV-S1/S3, and PICV-control without any TARV segment insertion) via oral route at 1 week of age. Birds in all groups were revaccinated with at 3 weeks of age via intranasal (I/N) route. Blood samples were collected at 3 and 5 weeks of age. At the end of the experiment, all birds were euthanized, and necropsy was done to detect the development of any gross lesions.

2.7 Serum neutralization assay: Sera were separated from the collected blood samples and subjected to serum neutralization assay against TARV-SKM121. Significant variations (P<0.05) in serum neutralization titers among different groups were tested by using non-parametric Kruskal Wallis test followed by Mann Whitney U test.

3. Results

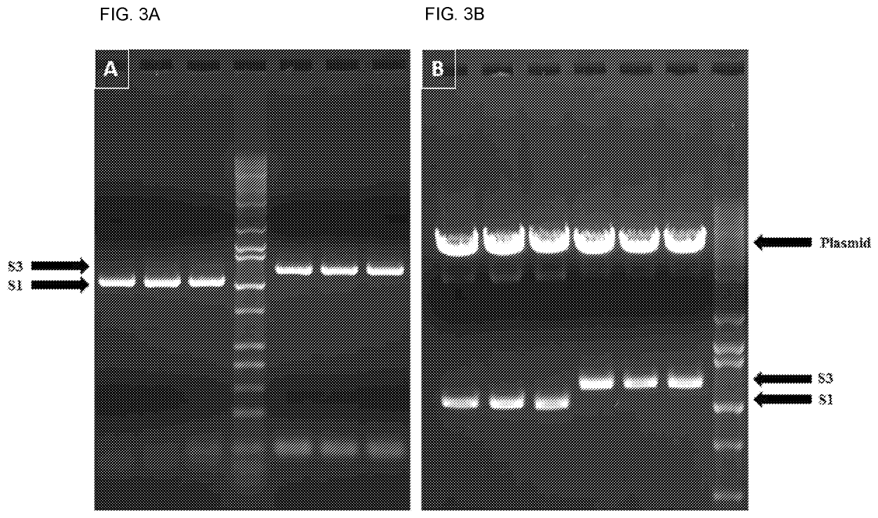

3.1 Cloning of reovirus genes into PICV plasmids: The RT-PCR amplification of 51 and S3 ORF yielded the expected product sizes of 1031 bp and 1157 bp, respectively (FIG. 3A). These products were gel purified and cloned into pP18S2-MCS/NP and pP18S1-GPC/MCS, respectively. Restriction enzyme double digestion confirmed the presence of reovirus genes in recombinant PICV plasmids (FIG. 3B). Sanger sequencing confirmed the absence of mutation in cloned viral gene as well as their correct reading frame and correct orientation in the vector backbones (data not shown).

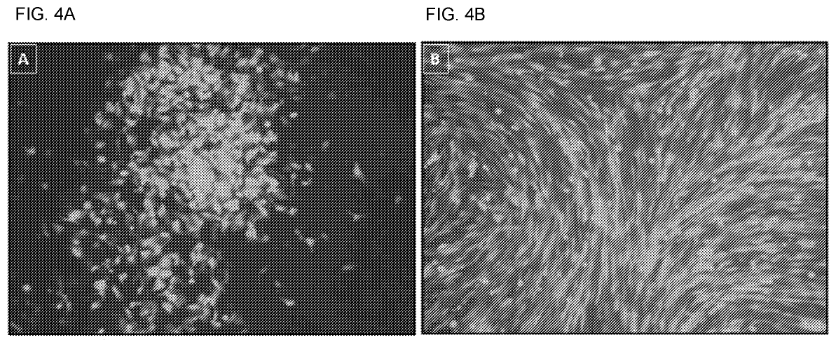

3.2 Plasmid transfection and virus rescue: Viable recombinant PICVs were rescued successfully following transfection of BSRT7-5 cells with the three plasmids in various combinations as shown in Table 2. The GFP expression was observed 48-72 h post transfection in cells transfected with at least one GFP-containing plasmid (FIG. 4A) (all monovalent vaccines in Table 2). The GFP-expressing foci increased in size over the time course of transfection. The supernatants were collected from transfected BSR7-5 cells and were used to infect BHK21 cells. Strong GFP expression was detected in infected BHK21 cells at 24-48 hpi using fluorescence microscopy (FIG. 4B) indicating the rescue of viable viruses. At every rescue attempt, we obtained infectious viruses at 48-72 h post transfection. The recombinant viruses showed minor GFP fluorescence in QT-35 and LMH cells at 96 hours after inoculation. As expected, the bivalent viruses having two TARV genes on both plasmids did not produce any green fluorescence.

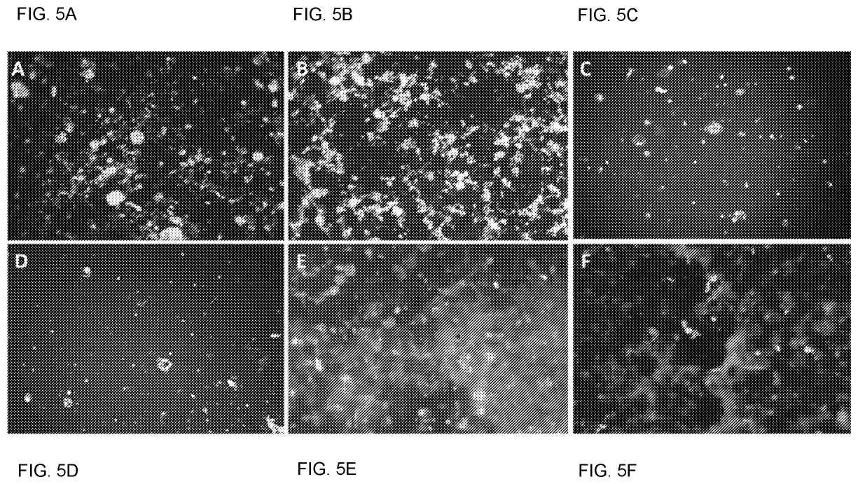

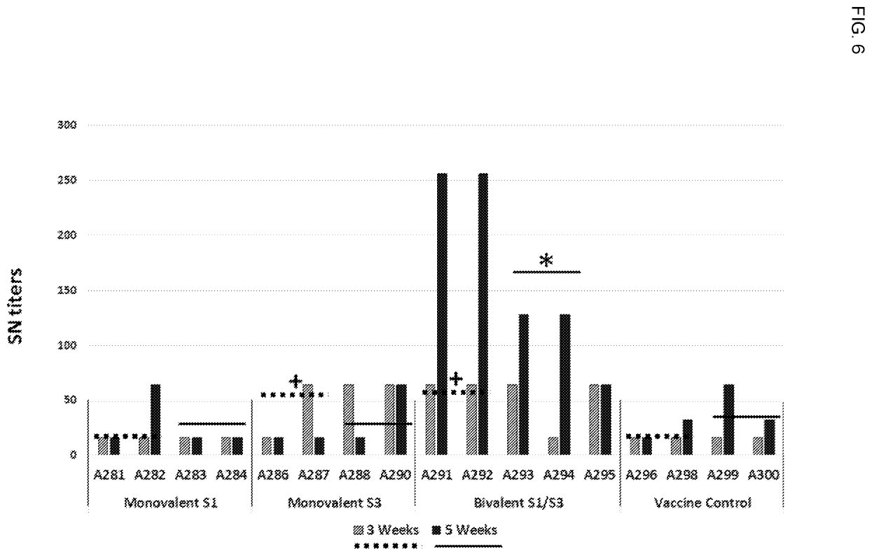

3.3 Recombinant PICVs expressing reovirus antigens: Strong GFP expression by infected BHK21 cells indicated successful rescue of recombinant viruses. The supernatant from infected BHK21 cells (passage P1, P2 and up to P3) was used to detect reovirus genes by RT-PCR. The results confirmed the presence of both viral genes in bivalent vaccine viruses and either 51 or S3 gene in the monovalent vaccine viruses. To verify the expression of reovirus antigenic proteins (GC and GB) by the recombinant PICVs, we infected BHK21 cells with transfection supernatant and then at 48 hpi conducted a direct fluorescence assay (DFA) using polyclonal FITC-conjugated anti-avian reovirus antibodies. The PICVs grown on BHK-21 showed varying degrees of fluorescence (FIG. 5). The monovalent and bivalent PICVs that contained either 51 or S3 or both, showed fluorescence in BHK-21 cells. Although we did not quantify the amount of fluorescence, PICVs containing SKM121 gene segments showed a remarkably higher degree of fluorescence, particularly the bivalent PICV that contained codon optimized S1 and S3 segments (FIG. 6). Minimal fluorescence was observed in negative controls (cells that contained recovered PICV vector without any TARV segment).

3.5 Vaccination/Necropsy: Birds inoculated with the vaccines neither displayed any clinical disease or illness during the study nor showed any gross lesions at necropsy.

3.6 Serum Neutralization Assay: At 3 weeks of age, 3 of 4 sera from birds inoculated with monovalent PICV-S3 vaccine and 4 of 5 sera from birds inoculated with bivalent PICV-S1/S3 vaccine showed serum neutralizing (SN) antibody titers of 64, which were significantly higher (p<0.05) than the other two groups (FIG. 6). At 5 weeks of age, only one of three birds inoculated with monovalent PICV-S1, monovalent PICV-S3 and PICV-control had an SN antibody titer of 64 while all five sera from birds inoculated with the bivalent vaccine had high SN antibody titers ranging from 64 to 256, which were significantly higher (p<0.05) than the other three groups (FIG. 6). Individual bird's data show that there was a remarkable increase in the SN antibody titers of birds inoculated with the bivalent PICV-S1/S3 vaccine at 5 weeks of age while other groups did not show any remarkable increase in titers (FIG. 6).

4. Discussion

Using PICV as a vector to express TARV antigenic proteins appears an attractive alternative to the use of live attenuated vaccines that may result in the emergence of mutant strains. Recombinant PICV expressing the hemagglutinin and nucleoprotein genes of influenza initiated humoral and cell-mediated immune responses and provided full protection against lethal influenza in mice [7] and was shown to be safe and effective in chickens. The purpose of this study was to develop recombinant PICV-TARV vaccines that can carry S1 and/or S3 genes of turkey arthritis reoviruses. We successfully recovered PICV after insertion of TARV genomic segments in the PICV plasmids, detected the inserted genes and confirmed the expression of recombinant antigenic proteins.

A total of 12 different recombinant PICVs were developed. We used the wild type genes from three different TARV isolates in addition to codon-optimized genes from one TARV isolate in an effort to find an optimum TARV candidate to be used in developing a vaccine. The recovered PICV recombinants grew well in BHK-21 cells but showed minimal growth on QT-35 and LMH cells as determined by the expression of green fluorescence protein in recombinant PICV that contained one each of TARV gene and GFP gene. No GFP gene was inserted in the double recombinant PICV (containing both S1 and S3 genes of TARV) and hence they could not be subjected to the green fluorescence test. We assume that these viruses grew the same way as the monovalent viruses because their plasmids and transfection were done under the same conditions. The detection of the inserted genes and protein expression was helpful in determining successful recovery and growth of bivalent PICV. The use of RT-PCR to detect the inserted TARV genome in recovered viruses helped in determining the success of recovering recombinant PICV that contained either S1 or S3, or both.

To confirm the expression of reoviral antigenic proteins by the recombinant PICVs, we used the direct fluorescent antibody technique (FA) using FITC-conjugated anti-avian reovirus antibodies. The FA procedure included dehydration and long fixation with acetone. These two steps helped eliminate GFP fluorescence and increased permeabilization of the cell membrane. The permeabilization of the cell membrane enabled the entrance of the FITC-conjugated antibody to the intracellular recombinant viral proteins. Although we did not have a quantitative method to measure the amount of the expressed protein, we could subjectively observe that the fluorescence produced by viruses recombined with SKM121 genes was more than that produced by SKM73 and SKM95. The bivalent PICV that contained codon optimized S1 and S3 of SKM121 showed the best fluorescence indicating the growth of these viruses, which did not have any GFP fluorescence after transfection.

Since codon-optimized PICV-based TARV SKM121 vaccines showed the best expression of TARV S1 and/or S3 proteins, we used these vaccines for a pilot in vivo experiment. PICV without an insertion of TARV gene was used as a control. The in vivo experiment was to determine the safety and humoral response generated by the recombinant vaccines in turkey poults. Turkey poults were vaccinated with the same dose of vaccine (0.3 mL, 3×10⁵ PFU/mL) via oral and intranasal route using a prime-boost strategy. Poults were primed at 1 week of age because birds are susceptible to avian reovirus infection in the early days of their life [13,14]; hence, vaccination strategies are designed to provide passive immunity from maternal antibodies by vaccinating breeders or by actively immunizing young bids with a live vaccine [15]. Priming at 1 week of age was also considered to avoid vaccination shock and poor intestinal immunity in day old birds [15]. Booster dose was given intranasally at 3 weeks of age, targeting the coarse spray administration with the same vaccine as previously described [16]. The booster vaccination was done after 2 weeks of priming because the recombinant PICV-based vaccine needs 2-3 weeks to provoke the best immune responses. Immunogenicity of the codon-optimized bivalent PICV-based TARV SKM121 vaccine was demonstrated by the production of high SN antibody titers. In future in vivo experiments, we plan to further characterize the effect of the dose of the recombinant PICV-based TARV vaccines on antibody titer and response to reovirus challenge in turkeys, as well as to study the efficacy of protection of the PICV-based TARV vaccines against heterologous and homologous challenge viruses. In conclusion, the bivalent PICVs carrying codon-optimized genes of the SKM121 turkey reoviral isolate showed the best results in vitro and in vivo. We plan to use PICV carrying the codon-optimized genes of SKM121 for future in vivo studies in turkeys to study the efficacy of protection of the PICV-TARV recombinant vaccines against heterologous and homologous challenge viruses.

### Citations for Example 1

[1] Mor S K, Sharafeldin T A, Porter R E, Ziegler A, Patnayak D P, Goyal S M. Isolation and characterization of a turkey arthritis reovirus. Avian Dis 2013; 57:97-103.
[2] Sharafeldin T A, Mor S K, Bekele A Z, Verma H, Goyal S M, Porter R E. The role of avian reoviruses in turkey tenosynovitis/arthritis. Avian Pathol 2014; 43:371-378.
[3] Sharafeldin T A, Mor S K, Bekele A Z, Verma H, Noll S L, Goyal S M, Porter R E. Experimentally induced lameness in turkeys inoculated with a newly emergent turkey reovirus. Vet Res 2015; 46:11.
[4] Benavente J, Martinez-Costas J. Avian reovirus: structure and biology. Virus Res 2007; 123: 105-119.
[5] Varela R, Benavente J. Protein coding assignment of avian reovirus strain S1133. J Virol 1994; 68: 6775-6777.
[6] Wickramasinghe R, Meanger J, Enriquez C E, Wilcox G E. Avian reovirus proteins associated with neutralization of virus infectivity. Virology 1993; 194: 688-696.
[7] Dhanwani R, Zhou Y, Huang Q, Verma V, Dileepan M, Ly H et al. A novel live Pichinde virus-based vaccine vector induces enhanced humoral and cellular immunity upon a booster dose. J Virol 2016; 90:2551-2560.
[8] Trapido H, Sanmartin C. Pichinde virus, a new virus of the Tacaribe group from Colombia. Am J Trop Med Hyg 1971; 20:631-641.
[9] Emonet S F, Garidou L, McGavern D B, de la Torre J C. Generation of recombinant lymphocytic choriomeningitis viruses with trisegmented genomes stably expressing two additional genes of interest. Proc Natl Acad Sci 2009; 106:3473-3478
[10] Flatz L, Hegazy A N, Bergthaler A, Verschoor A, Claus C, Fernandez M et al. Development of replication-defective lymphocytic choriomeningitis virus vectors for the induction of potent CD8+ T cell immunity. Nat Med 2010; 16:339-345
[11] Popkin D L, Teijaro J R, Lee A M, Lewicki H, Emonet S, de la Torre J C et al. Expanded potential for recombinant trisegmented lymphocytic choriomeningitis viruses: protein production, antibody production, and in vivo assessment of biological function of genes of interest. J Virol 2011; 85:7928-7932
[12] Ortiz-Riano E, Cheng B Y, Carlos de la Torre J, Martinez-Sobrido L. Arenavirus reverse genetics for vaccine development. J Gen Virol 2013; 94:1175-1188
[13] Jones, R. C.; Georgiou, K. Reovims-induced tenosynovitis in chickens: The influence of age at infection. Avian Pathol. 1984, 13, 441-457.
[14] Roessler, D. E.; Rosenberger, J. K. In vitro and in vivo characterisation of avian reovirus. III. Host factors affecting virulence and persistence. Avian Dis. 1989, 33, 555-565.
[15] Jones, R. C. Avian reovirus infections. Rev. Sci. Tech. Off. Int. Epizoot. 2000, 19, 614-619.
[16] Giambrone, J. J.; Hathcock, T. L. Efficacy of coarse-spray administration of a reovirus vaccine in young chicks. Avian Dis. 1991, 35, 204-209.

### Example 2

Efficacy of a Recombinant Viral Vectored Vaccine Against Turkey Arthritis Reovirus

Objective: To evaluate the safety and efficacy of a bivalent Pichinde virus-vectored vaccine (rPICV-TARV) expressing the immunogenic Sigma C and Sigma B proteins of the turkey arthritis reovirus (TARV) strain SKM121 in turkey poults.

Experimental plan: Day-old turkey poults (n=80) were procured from a non-vaccinated and reovirus-free flock. Ten poults were euthanized on the day of arrival to collect serum samples for anti-TARV antibody testing, which were found to be negative. Meconium, intestine and tendon samples were tested by real time RT-PCR (rRT-PCR) to ensure that these turkey poults were also negative for reovirus. The remaining 70 poults were randomly divided into five groups (see Table 3 below). These groups were (1) negative control (NC), (2) vaccine control (VC), (3) vaccinated and challenged with TARV SKM121 (V-SKM), (4) sentinels placed in contact with vaccinated birds and then challenged with TARV SKM121 (Sen-SKM), (5), non-vaccinated birds challenged with TARV SKM121 (SKM) as positive controls to ensure that the challenge virus was infectious. The five groups of birds were housed in separate air-filtered isolators. Food and water were supplied ad libitum.

Poults were vaccinated orally with a primary dose of rPICV-TARV vaccine (0.2 ml, 3×10⁷ PFU/ml) at 2 days of age (day of age). In Sen-SKM group, 12 birds were wing banded and added as sentinels after 2 days of primary vaccination (4 day of age). Poults in groups 2, 3, and 4 were boosted intranasally (except the sentinels and NC) with 0.2 ml (3×10⁷ PFU/ml) of the vaccine at 9 days of age. On day 14, blood samples were collected from the non-vaccinated, vaccinated, and sentinel birds (groups 1, 3, and 4) for serology. At 15 days of age, birds in groups 3, 4, and 5 were challenged orally with 0.2 ml (3.2×10⁷ TCID₅₀/ml) of TARV SKM121 while groups 1 and 2 (NC and VC) were sham inoculated with 0.2 ml of cell culture media (MEM). The birds were examined daily for any overt clinical signs or mortality. No mortality or lameness was observed in any of the birds. All birds were euthanized at the age of 35 days and their body weights were recorded. We chose to euthanize the birds at 35 days of age because our previous research to establish a TARV challenge model has indicated that poults orally challenged at one week of age with a virulent strain of TARV did not show histological evidence of tenosynovitis until after 28-35 days of age. At necropsy, gross lesions were noted followed by collection of ileocecum and hock joint with gastrocnemius and digital flexor tendons for real time RT-PCR (rRT-PCR) and histopathology.

TABLE 3
Experimental plan showing the five groups of birds.

| | | | Day of age at which the birds were given the | | | | |
|---|---|---|---|---|---|---|---|
| | | Group | indicated treatment | | | | |
| | | description | | Sentinel | | | Euthanasia |
| Group | Group | (number of | Oral | birds | Booster | | and |
| No. | Name | birds) | Vac | added | Vac | Challenge | sampling |
| 1 | NC (negative | Neg ctrl (12) | — | — | — | — | 35 |
| | control) | | | | | | |
| 2 | VC (vaccine | V-ONLY (12) | 2 | — | 9 | — | 35 |
| | control) | | | | | | |
| 3 | V-SKM | V-Ch- | 2 | — | 9 | 15 | 35 |
| | (vaccinated | SKM121 (12) | | | | | |
| | challenged) | | | | | | |
| 4 | Sen-SKM | V + Sent-Ch- | 2 | 4 | 9 | 15 | 35 |
| | (sentinel | SKM121 | | | | | |
| | challenged) | (10 + 12) | | | | | |
| 5 | SKM | Ch- SKM121 | — | — | — | 15 | 35 |
| | (challenge | (12) | | | | | |
| | only) | | | | | | |

Summary of Results:

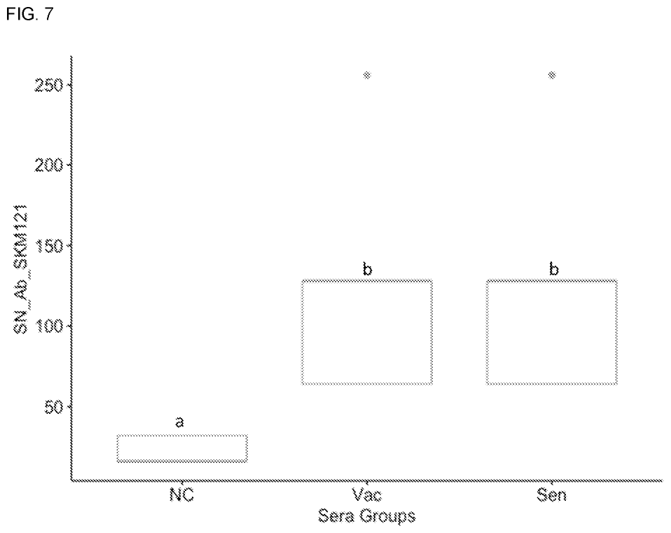

Poults directly vaccinated with rPICV-TARV and the sentinel poults (in contact with vaccinated poults) produced serum neutralizing antibodies against TARV (FIG. 7). The sentinel poults developed neutralizing antibodies at the same level as the vaccinated poults (antibody titers 1:64 to 1:256) indicating that the vaccine can be transmitted to non-vaccinated pen mates (sentinels) followed by the development of a humoral immune response. This is an unexpected but positive outcome, because it suggests that the rPICV-TARV vaccine can be used to mediate population (or herd) immunity in turkey flocks.

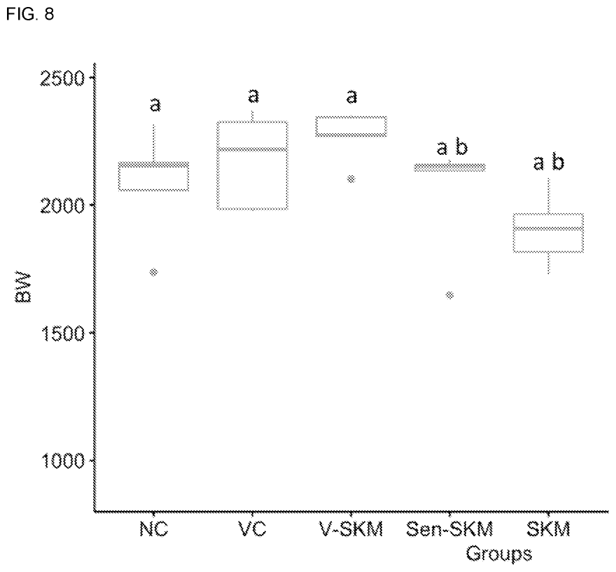

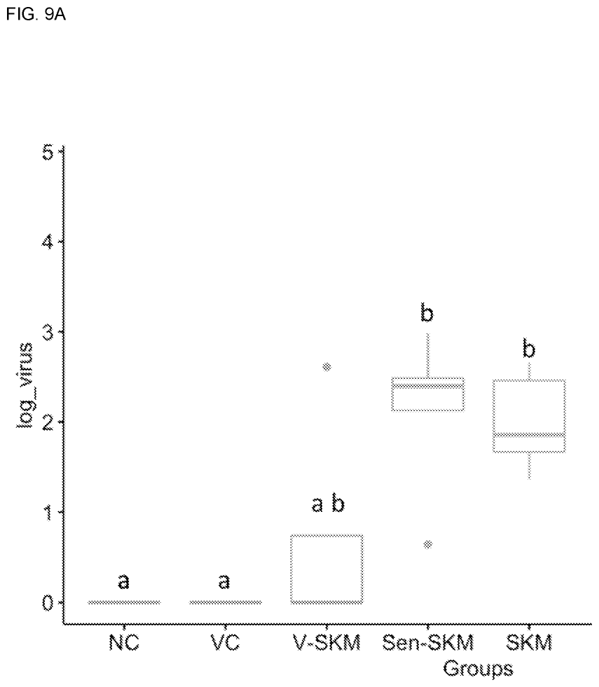

The vaccinated and control poults showed similar body weights, indicating the good safety profile of this type of a vaccine in turkey as no adverse symptoms or negative effects on feed efficiency of the vaccinated poults were observed. In contrast, the mean body weights of poults challenged with the virulent reovirus alone were significantly lower than that of the vaccinated poults (V-SKM) at 35 day of age, indicating the expected virulent nature of the challenge virus used in the study (FIG. 8).

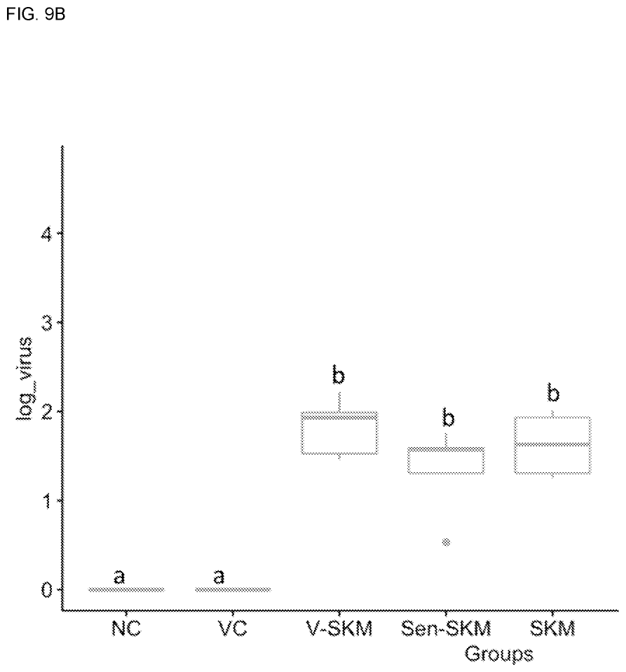

At 35 days of age, the vaccinated poults had significantly lower reoviral replication (TARV-SKM) in the intestine compared to that in non-vaccinated/challenged poults, indicating that the rPICV-TARV vaccine could suppress replication of an infectious reovirus challenge in the intestine of these animals (FIG. 9A). However, at 35 days of age, there were no significant differences in TARV-SKM replication in the gastrocnemius tendons of rPICV-TARV-vaccinated poults, sentinel poults and nonvaccinated/challenged poults (FIG. 9B). This observation is unexpected and suggests an area for further investigations.

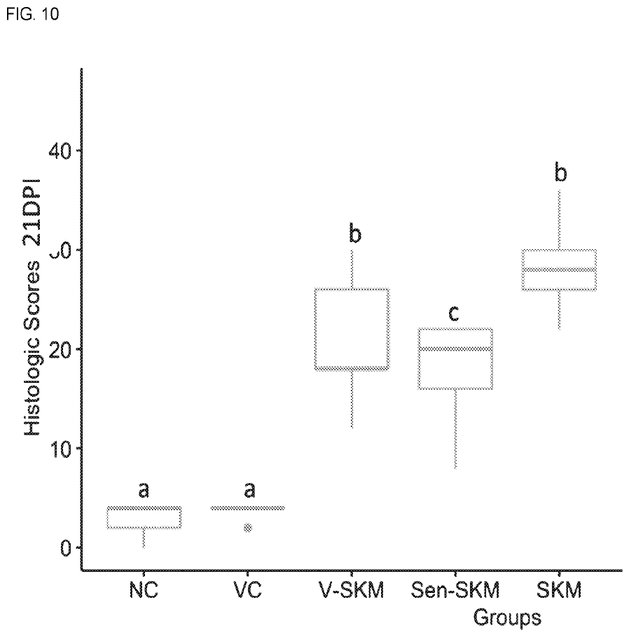

At 35 days of age, the vaccinated/challenged poults had numerically greater mean tendon inflammation than vaccinated and sentinel poults; however, this difference was statistically significant only in the sentinel poults (FIG. 10). Again, this observation is unexpected and suggests another potential area for further investigations.

Conclusions and Discussion

The study produced some expected and unexpected findings. First, the rPICV-TARV vaccine can produce serum neutralizing antibodies against TARV after dual dosing of turkey poults. Additionally, the vaccinated poults can transmit the virus laterally to sentinels, resulting in a similar production of serum neutralizing antibodies against TARV and suggesting a potential advantage and utility of this formulation of the vaccine to produce population (or herd) immunity. Vaccinated/challenged poults had no mortality and similar body weights to negative control poults, indicating the rPICV-TARV vaccine was safe. Vaccinated poults likely maintain their body weights because reoviral replication in the intestine is reduced post challenge compared to that in non-vaccinated poults. At the current dose and vaccination timing, the rPICV-TARV vaccine does not appear to effectively reduce TARV replication in the gastrocnemius tendon when compared to the vaccinated poults. Alternatively, there might be a potential tissue-specificity phenomenon of reovirus replication and vaccine efficacy for future investigations. Future work should focus on adjustment of the vaccine regimen for rPICV-TARV to provide a broad level of protection and to examine the potential of reovirus replication and vaccine efficacy in a certain and specific tissue of the vaccinated animals. Future work should also focus on altering the age of inoculation, the vaccine dose, increasing the time period between the prime and boost in order to enhance humoral immune development and maintenance in the vaccinated animals, and increasing the numbers of poults used in each experimental group. The period between prime and booster vaccination in our study was only 7 days; however, our recent studies have indicated that the rPICV-TARV vaccine should be given at least three weeks prior to virus challenge in order to allow the vaccinated animals to form memory B cells for a heightened level of immune reaction to the antigens and antibody production. The age at prime-boost (02 and 09 day of age) and age at virus challenge (15 day of age) adopted in this study were based on previous research conducted on reovirus infection in chickens with a particular attention to the age of chicken susceptibility to reovirus infection. However, our recent study (Example 1 and Kumar et al., 2021 Pathogens. 10(2):197. doi: 10.3390/pathogens10020197. PMID: 33668435) recommended a gap of 2-3 weeks between the prime and booster dose in turkeys. The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A genetically engineered Pichinde virus comprising:
three ambisense genomic segments,
wherein the first genomic segment comprises a coding region encoding a Z protein and a coding region encoding a L RNA-dependent RNA polymerase (L RdRp) protein,
wherein the second genomic segment comprises a coding region encoding a nucleoprotein and a coding region, wherein the coding region encodes a first avian reovirus protein,
wherein the third genomic segment comprises a coding region encoding a glycoprotein and a coding region, wherein the coding region encodes a second avian reovirus protein, and
wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

2-3. (canceled)

4. An infectious virus particle comprising the three genomic segments of claim 1.

5. A composition comprising the isolated infectious virus particle of claim 4.

6. A collection of vectors comprising:
a first vector encoding a first genomic segment comprising a coding region encoding a Z protein and a coding region encoding a L RdRp protein, wherein the first genomic segment is antigenomic,
a second vector encoding a second genomic segment comprising a coding region encoding a nucleoprotein and a coding region encoding a first avian reovirus protein, wherein the second genomic segment is antigenomic, and
a third vector encoding a third genomic segment comprises a coding region encoding a glycoprotein and a coding region, wherein the coding region encodes a second avian reovirus protein, wherein the third genomic segment is antigenomic,
wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

7. The collection of claim 6 wherein the vectors are plasmids.

8. The collection of claim 7 wherein the plasmids further comprise a T7 promoter.

9-14. (canceled)

15. A reverse genetics system for making a genetically engineered virus comprising three vectors,
wherein a first vector encodes a first genomic segment comprising a coding region encoding a Z protein and a coding region encoding a L RdRp protein, wherein the first genomic segment is antigenomic,
wherein the second vector encodes a second genomic segment comprising a coding region encoding a nucleoprotein and a coding region encoding a first avian reovirus protein, wherein the second genomic segment is antigenomic,
wherein the third vector encodes a third genomic segment comprises a coding region encoding a glycoprotein and a second avian reovirus particle, wherein the third genomic segment is antigenomic,
wherein the first avian reovirus protein is reovirus sigma C protein and the second avian reovirus protein is reovirus sigma B protein, or the first avian reovirus protein is reovirus sigma B protein and the second avian reovirus protein is reovirus sigma C protein.

16. The reverse genetics system of claim 15 wherein each plasmid comprises a T7 promoter.

17. A method for using a reverse genetics system, comprising:
introducing into a cell the three vectors of genomic segments of claim 15; and
incubating the cell under conditions suitable for the transcription of the three genomic segments and expression of the coding regions of each genomic segment.

18. The method of claim 17 wherein the cell produces virus particles, the method further comprising isolating virus particles produced by the cell, wherein each virus particle comprises the three genomic segments.

19. The method of claim 18 wherein the virus particles are infectious.

20. (canceled)

21. The method of claim 17 wherein the introducing comprises contacting the cell with a virus particle comprising the three genomic segments.

22. The method of claim 17 wherein the cell is ex vivo.

23. The method of claim 17 wherein the cell is a vertebrate cell.

24. The method of claim 23 wherein the vertebrate cell is an avian cell.

25. The method of claim 24 wherein the avian cell is a chicken embryonic fibroblast.

26. A method for treating a subject, comprising
administering to a subject the infectious virus particle of claim 4 to result in an immune response, wherein the subject is at risk of infection by a reovirus.

27. (canceled)

28. The method of claim 26 wherein the subject is avian.

29. The method of claim 28 wherein the avian is a turkey or chicken.

30. The method of claim 26 wherein the immune response comprises a humoral immune response.

31. (canceled)
